(19) United States
(12) Patent Application Publication
(10) Pub. No.: US 2023/0192837 A1
(43) Pub. Date: Jun. 22, 2023

(54) HUMAN TUMOR NECROSIS FACTOR ALPHA ANTIBODIES

(71) Applicant: Eli Lilly and Company, Indianapolis, IN (US)

(72) Inventors: Grace Chao, San Diego, CA (US); Yiqing Feng, Carmel, IN (US); Donmienne Doen Mun Leung, San Diego, CA (US); Songqing Na, San Diego, CA (US); Bharathi Ramamurthy, San Diego, CA (US); Jianghuai Xu, San Diego, CA (US)

(21) Appl. No.: 18/054,284

(22) Filed: Nov. 10, 2022

Related U.S. Application Data
(60) Provisional application No. 63/278,245, filed on Nov. 11, 2021.

(51) Int. Cl. C07K 16/24; A61K 47/68; A61P 37/06

(52) U.S. Cl. CPC C07K 16/241; A61K 47/6845; A61P 37/06; A61K 2039/505

(57) ABSTRACT

The present disclosure relates to antibodies that specifically bind soluble and membrane forms of human TNFα, compositions comprising such TNFα antibodies, and methods of using such TNFα antibodies and compositions.

### SEQUENCE LISTING FILE

The present application is being filed along with a Sequence Listing in ST.26 XML format. The Sequence Listing is provided as a file titled “X30122_SequenceListing” created Sep. 20, 2022 and is 57 kilobytes in size. The Sequence Listing information in the ST.26 XML format is incorporated herein by reference in its entirety.

The present disclosure is in the field of medicine. Particularly the present disclosure relates to antibodies that bind soluble and membrane forms of human Tumor Necrosis Factor Alpha (TNFα), compositions comprising such TNFα antibodies, and methods of using such TNFα antibodies and compositions.

Chronic autoinflammatory immune disorders arise from the body's production of an immune response against its own tissue. Excessive and prolonged activation of immune cells, such as T and B lymphocytes, and overexpression of pro-inflammatory cytokine TNFα, together with other mediators such as interlukin-6 (IL-6), interluekin-1 (IL-1), and interferon gamma (IFN-γ), play a central role in the pathogenesis of autoinflammatory immune responses.

Tumor Necrosis Factor alpha (also known as TNFα, tumor necrosis factor, TNF, Cachectin) is a pleiotropic homotrimeric cytokine reported to be secreted by activated macrophages, monocytes, CD4⁺ and CD8⁺ T lymphocytes, natural killer (NK) cells, B cells, neutrophils, and endothelial cells. TNFα is expressed in both a soluble and a membrane form (the membrane-bound precursor form can be proteolytically cleaved into a soluble homotrimer by metalloproteinase TNF alpha converting enzyme (TACE)). The soluble TNFα (sTNFα) facilitates various biological activities through type 1 receptors (TNFR1, also known as TNFRSF1A, CD120a, and p55) and type 2 receptors (TNFR2, also known as TNFRSF1B, CD120b, and p′75). TNFα binds to its receptors, mainly TNFR1 and TNFR2, and transmits molecular signals for biological functions such as inflammation and cell death. TNFRs are activated by both sTNFα and transmembrane TNFα (tmTNFα). TNFα plays a role in the regulation of immune cells and is associated with chronic inflammation, specifically in acute phase inflammatory reactions. Excess amounts of TNFα have been associated with various chronic autoinflammatory immune disorders.

Anti-TNFα therapeutics targeting chronic autoinflammatory immune disorders are known, and either approved or in clinical development. Such therapeutics include, adalimumab, infliximab, golimumab, certolizumab, and Etanercept. (Jang, D-i., Int. J. Mol. Sci., 2021, 22(5): 2719). However, a major shortcoming of their use is the development of anti-drug antibodies in some patients receiving the anti-TNFα therapeutic. Such anti-drug antibodies may be non-neutralizing antibodies that bind to the anti-TNFα therapeutic simultaneously with TNFα, or they may be neutralizing antibodies which reduce the effective concentrations of the anti-TNFα therapeutic in the serum and/or compete with TNFα for the antigen-binding site (paratope) thus inhibiting the working mechanism of the anti-TNFα therapeutic. (Schie K A, et al, Annals of the Rheumatic Diseases, 2015, 74: 311-314). For example, studies have shown that greater than ninety percent of anti-TNFα drug antibodies are neutralizing and may be cross reactive to other anti-TNFα antibody therapeutics. (Schie K A, et al, 2015). As such, in some instances, patients developing anti-drug antibodies to anti-TNFα therapeutics have been reported to have, diminished clinical response to these therapeutics and/or adverse events such as infusion related reactions characterized by symptoms such as fever, pruritus, bronchospasms, or cardiovascular collapse during or within the first day after drug administration (Atiqi, S., Front Immunol., 2020, 26(11): 312). Thus, anti-drug antibody responses can render patients with limited treatment options.

There, thus, remains a need for alternative anti-TNFα therapeutics that neutralize soluble and membrane TNFα with desirable affinity, provide a sustained duration of action, and are capable of treating chronic autoinflammatory immune disorders. Particularly, there remains a need for an anti-human TNFα antibody that has a reduced risk of eliciting an anti-drug antibody response and/or does not significantly bind to anti-drug antibodies against other anti-TNFα antibody therapeutics. There further remains a need for an anti-human TNFα antibody that is capable of treating chronic autoinflammatory immune disorders and treating chronic autoinflammatory immune disorders in patients who have developed anti-drug antibody responses to treatment with other TNFα therapeutics. Such anti-human TNFα antibodies will preferably also possess low immunogenicity risk, and/or good developability profiles such as good physical-chemical properties to facilitate development, manufacturing, and formulation.

### Written Description

The present disclosure provides anti-human TNFα antibodies that bind and neutralize human TNFα, and inhibit TNF receptor mediated responses (e.g., NFkB activation, cytokine production). The present disclosure further provides compositions comprising such anti-human TNFα antibodies and methods of using such anti-human TNFα antibodies and compositions. Particularly, the present disclosure provides anti-human TNFα antibodies that have desirable binding affinities, bind and neutralize soluble and membrane human TNFα, internalize upon binding to membrane TNFα, and/or exhibit low to no binding to anti-drug antibodies against other anti-TNFα therapeutics, and have potential for use in the treatment of patients with chronic autoinflammatory immune disorders who have developed anti-drug antibodies to prior treatment with an anti-TNFα therapeutic, e.g., adalimumab. Such chronic autoinflammatory immune disorders include Rheumatoid Arthritis (RA), Juvenile Idiopathic Arthritis, Psoriatic Arthritis (PsA), Ankylosing Spondylitis (AS), Crohn's Disease (CD), Ulcerative Colitis (UC), Plaque Psoriasis (PS), Hidradenitis Suppurativa (HS), Uveitis, non-infectious Intermediate, Posterior or Pan Uveitis, or Behcet's Disease. The anti-human TNFα antibodies as disclosed herein, further present low immunogenicity risk and/or good developability profiles such as good physical-chemical properties (e.g., viscosity, aggregation, stability) to facilitate development, manufacturing, and formulation. As such, the anti-human TNFα antibodies provided herein have one or more of the following properties: 1) bind human TNFα with desirable binding affinities, 2) bind rhesus macaque monkey, and/or canine TNFα with desirable binding affinities, 3) inhibit TNFR mediated signaling (e.g., NFkB), 4) inhibit cytokine production (e.g., CXCL1) in vivo, 5) have low to no binding to anti-drug antibodies against other anti-TNFα therapeutic, e.g., adalimumab, 6) internalize when bound to membrane TNFα, 7) have low immunogenicity risk, and/or 8) have good developability profiles such as having acceptable viscosity, and/or aggregation profile to facilitate development, manufacturing, and formulation.

In some embodiments, the present disclosure provides an antibody that binds human TNFα, and comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 3, the LCDR1 comprises SEQ ID NO: 4, the LCDR2 comprises SEQ ID NO: 5, and the LCDR3 comprises SEQ ID NO: 6. In some embodiments, the antibody comprises a VH comprising SEQ ID NO: 7 and a VL comprising SEQ ID NO: 8. In some embodiments, the antibody comprises a heavy chain (HC) comprising SEQ ID NO: 9 and a light chain (LC) comprising SEQ ID NO: 10.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
a. the HCDR1 comprises SEQ ID NO: 22;
   the HCDR2 comprises SEQ ID NO: 23;
   the HCDR3 comprises SEQ ID NO: 13;
   the LCDR1 comprises SEQ ID NO: 4, 14, or 46;
   the LCDR2 comprises SEQ ID NO: 5; and
   the LCDR3 comprises SEQ ID NO: 6;
b. the HCDR1 comprises SEQ ID NO: 22;
   the HCDR2 comprises SEQ ID NO: 23;
   the HCDR3 comprises SEQ ID NO: 13;
   the LCDR1 comprises SEQ ID NO: 14;
   the LCDR2 comprises SEQ ID NO: 5; and
   the LCDR3 comprises SEQ ID NO: 15 or 47;
c. the HCDR1 comprises SEQ ID NO: 1;
   the HCDR2 comprises SEQ ID NO: 2;
   the HCDR3 comprises SEQ ID NO: 30;
   the LCDR1 comprises SEQ ID NO: 31;
   the LCDR2 comprises SEQ ID NO: 5; and
   the LCDR3 comprises SEQ ID NO: 32; or
d. the HCDR1 comprises SEQ ID NO: 1;
   the HCDR2 comprises SEQ ID NO: 2;
   the HCDR3 comprises SEQ ID NO: 13;
   the LCDR1 comprises SEQ ID NO: 14;
   the LCDR2 comprises SEQ ID NO: 5; and
   the LCDR3 comprises SEQ ID NO: 15.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 13, the LCDR1 comprises SEQ ID NO: 14, the LCDR2 comprises SEQ ID NO: 5, and the LCDR3 comprises SEQ ID NO: 15. In some embodiments, the antibody comprises a VH comprising SEQ ID NO: 16 and a VL comprising SEQ ID NO: 17. In some embodiments, the antibody comprises a HC comprising SEQ ID NO: 18 and a LC comprising SEQ ID NO: 19.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 4, the LCDR2 comprises SEQ ID NO: 5, and the LCDR3 comprises SEQ ID NO: 6. In some embodiments, the human TNFα antibodies comprise a VH comprising SEQ ID NO: 24 and a VL comprising SEQ ID NO: 8. In some embodiments, the antibody that binds human TNFα, comprises a heavy chain (HC) comprising SEQ ID NO: 25 and a light chain (LC) comprising SEQ ID NO: 10.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 15. In some embodiments, the antibody comprises a VH comprising SEQ ID NO: 24 and a VL comprising SEQ ID NO: 17. In some embodiments, the antibody comprises a heavy chain (HC) comprising SEQ ID NO: 25 and a light chain (LC) comprising SEQ ID NO: 19.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 6. In some embodiments, the antibody comprises a VH comprising SEQ ID NO: 24 and a VL comprising SEQ ID NO: 27. In some embodiments, the comprises a heavy chain (HC) comprising SEQ ID NO: 25 and a light chain (LC) comprising SEQ ID NO: 28.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 46, the LCDR2 comprises SEQ ID NO: 5, and the LCDR3 comprises SEQ ID NO: 6. In some embodiments, SEQ ID NO: 46 comprises amino acid residues QASQGIXaa₇NYLN wherein Xaa₇ of SEQ ID NO: 46 is Serine or Arginine. In some embodiments, the antibody comprises a VH comprising SEQ ID NO: 24 and a VL comprising SEQ ID NO: 8. In some embodiments, the antibody comprises a VH comprising SEQ ID NO: 24 and a VL comprising SEQ ID NO: 27. In some embodiments, the antibody comprises a heavy chain (HC) comprising SEQ ID NO: 25 and a light chain (LC) comprising SEQ ID NO: 10. In some embodiments, the antibody comprises a heavy chain (HC) comprising SEQ ID NO: 25 and a light chain (LC) comprising SEQ ID NO: 28.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 47. In some embodiments, SEQ ID NO: 47 comprises amino acid residues QQYDXaa₅LPLT, wherein Xaa₅ of SEQ ID NO: 47 is Asparagine or Lysine. In some embodiments, the antibody comprises a VH comprising SEQ ID NO: 24 and a VL comprising SEQ ID NO: 17. In some embodiments, the antibody comprises a VH comprising SEQ ID NO: 24 and a VL comprising SEQ ID NO: 27. In some embodiments, the antibody comprises a heavy chain (HC) comprising SEQ ID NO: 25 and a light chain (LC) comprising SEQ ID NO: 19. In some embodiments, the antibody comprises a heavy chain (HC) comprising SEQ ID NO: 25 and a light chain (LC) comprising SEQ ID NO: 28.

In some embodiments, the present disclosure provides an antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 30, the LCDR1 comprises SEQ ID NO: 31, the LCDR2 comprises SEQ ID NO: 5, and the LCDR3 comprises SEQ ID NO: 32. In some embodiments, the antibody comprises a VH comprising SEQ ID NO: 33 and a VL comprising SEQ ID NO: 34. In some embodiments, the antibody that binds human TNFα, comprises a heavy chain (HC) comprising SEQ ID NO: 35 and a light chain (LC) comprising SEQ ID NO: 36.

In some embodiments of the present disclosure, the anti-human TNFα antibody is a fully human antibody. In some embodiments of the present disclosure, the anti-human TNFα antibody has a human IgG1 isotype.

In some embodiments of the present disclosure, the anti-human TNFα antibody has a modified human IgG1. In some embodiments, the modifications are in the heavy chain variable region (VH). In some embodiments, the modifications are in the light chain variable region (VL). In some embodiments, the modifications are in the VH and the VL. In further embodiments, the modified human IgG1 VH and/or VL provides a desirable viscosity profile and/or immunogenicity risk profile to the anti-human TNFα antibody of the present disclosure.

In further embodiments of the present disclosure, the anti-human TNFα antibody has a modified human IgG1 constant domain comprising engineered cysteine residues for use in the generation of antibody conjugate compounds (also referred to as bioconjugates) (see WO 2018/232088 A1). More particularly, in such embodiments of the present disclosure, the anti-human TNFα antibody comprises a cysteine at amino acid residue 124 (EU numbering), or a cysteine at amino acid residue 378 (EU numbering), or a cysteine at amino acid residue 124 (EU numbering) and a cysteine at amino acid residue 378 (EU numbering). Also provided herein, are antibody drug conjugates comprising the anti-human TNFα antibodies as disclosed herein.

In some embodiments of the present disclosure, the anti-human TNFα antibody binds soluble and membrane TNFα and inhibits binding of the TNFα to human TNF receptors (TNFR). In some embodiments of the present disclosure, the anti-human TNFα antibody binds soluble and membrane human TNFα and inhibits binding of human TNFα to human TNF receptors and inhibits TNFR mediated responses. In some embodiments, the anti-human TNFα antibody of the present disclosure inhibits binding of human TNFα to human TNFR and thus inhibits TNFR mediated responses such as: human TNFR activation, NFkB phosphorylation, cytokine production, and/or soluble and membrane TNFα induced cell killing. In some embodiments, the anti-human TNFα antibody of the present disclosure binds human TNFα and inhibits TNFR mediated NFkB phosphorylation and signal transduction on TNFR expressing cells by about 10%, about 20%, about 30%, about 40%, about 50%, about 60%, about 70%, about 80%, about 90%, or about 100%. In further embodiments, the anti-human TNFα antibody of the present disclosure binds human TNFα and inhibits TNFα-induced cytokine production (e.g., CXCL1), by about 10%, about 20%, about 30%, about 40%, about 50%, about 60%, about 70%, about 80%, about 90%, or about 100%. In yet further embodiments, the anti-human TNFα antibody of the present disclosure binds human TNFα and inhibits TNFα-induced cytokine production (e.g., CXCL1), by about 45% to about 95%. In further embodiments, the anti-human TNFα antibody of the present disclosure binds soluble human TNFα and inhibits TNFα induced cell killing by about 10%, about 20%, about 30%, about 40%, about 50%, about 60%, about 70%, about 80%, about 90%, or about 100%. In yet other embodiments, the anti-human TNFα antibody of the present disclosure binds membrane TNFα and inhibits membrane TNFα induced cell killing by about 10%, about 20%, about 30%, about 40%, about 50%, about 60%, about 70%, about 80%, about 90%, or about 100%.

In some embodiments, the anti-human TNFα antibody of the present disclosure binds membrane human TNFα and is internalized into the membrane TNFα expressing cells. In such embodiments, the antibody of the present disclosure binds membrane human TNFα and is internalized into the membrane TNFα expressing cells by about 10%, about 20%, about 30%, about 40%, about 50%, about 60%, about 70%, about 80%, about 90%, or about 100%.

In some embodiments, the anti-human TNFα antibody of the present disclosure has low to no binding to anti-drug antibodies against other anti-TNFα therapeutics (e.g., Adalimumab, Infliximab, Golimumab, Certolizumab, or Etanercept). In particular embodiments, the anti-human TNFα antibody of the present disclosure has low to no binding to anti-drug antibodies against Adalimumab. In such embodiments, the anti-human TNFα antibodies of the present disclosure may be used to treat patients who have developed anti-drug antibodies to prior treatment with other anti-TNFα therapeutic (e.g., Adalimumab) as defined herein. In further embodiments, the anti-human TNFα antibodies of the present disclosure may be used to treat patients who have developed anti-drug antibodies to other anti-TNFα therapeutics from prior treatment with such other anti-TNFα therapeutics and thus have a diminished clinical response or adverse reactions to the other anti-TNFα therapeutics. In such embodiments, the anti-human TNFα antibodies of the present disclosure have sufficiently different amino acid and nucleic acid sequences such that they have low to no binding to anti-drug antibodies against other anti-TNFα therapeutics. In particular embodiments, the anti-human TNFα antibodies of the present disclosure have sufficiently different CDR amino acid sequences such that they have low to no binding to anti-drug antibodies against other anti-TNFα therapeutics. In some embodiments, the other anti-TNFα therapeutic is Adalimumab, Infliximab, Golimumab, Certolizumab, or Etanercept.

In some embodiments, the present disclosure provides nucleic acids encoding a HC or LC, or a VH or VL, of the novel antibodies that bind human TNFα, or vectors comprising such nucleic acids.

In some embodiments, the present disclosure provides a nucleic acid comprising a sequence of SEQ ID NO: 11, 12, 20, 21, 26, 29, 37, or 38.

In some embodiments, nucleic acids encoding a heavy chain or light chain of the antibodies that bind human TNFα are provided. In some embodiments nucleic acids comprising a sequence encoding SEQ ID NO: 9, 10, 18, 19, 25, 28, 35, or 36 are provided. In some embodiments, nucleic acids comprising a sequence encoding an antibody heavy chain that comprises SEQ ID NO: 9, 18, 25, or 35 are provided. For example, the nucleic acid can comprise a sequence selected from SEQ ID NO: 11, 20, 26, or 37. In some embodiments, nucleic acids comprising a sequence encoding an antibody light chain that comprises SEQ ID NO: 10, 19, 28, or 36 is provided. For example, the nucleic acid can comprise a sequence selected from SEQ ID NO: 12, 21, or 29, or 38.

In some embodiments of the present disclosure, nucleic acids encoding a VH or VL of the antibodies that bind human TNFα are provided. In some embodiments, nucleic acids comprising a sequence encoding SEQ ID NO: 7, 8, 16, 17, 24, 27, 33, or 34 are provided. In some embodiments, nucleic acids comprising a sequence encoding an antibody VH that comprises SEQ ID NO: 7, 16, 24, or 33 are provided. In some embodiments, nucleic acids comprising a sequence encoding an antibody VL that comprises SEQ ID NO: 8, 17, 27, or 34 are provided.

Some embodiments of the present disclosure provide vectors comprising a nucleic acid sequence encoding an antibody heavy chain or light chain. For example, such vectors can comprise a nucleic acid sequence encoding SEQ ID NO: 9, 18, 25, or 35. In some embodiments, the vector comprises a nucleic acid sequence encoding SEQ ID NO: 10, 19, 28, or 36.

Provided herein are also vectors comprising a nucleic acid sequence encoding an antibody VH or VL. For example, such vectors can comprise a nucleic acid sequence encoding SEQ ID NO: 7, 16, 24, or 33. In some embodiments, the vector comprises a nucleic acid sequence encoding SEQ ID NO: 8, 17, 27, or 34.

Provided herein are also vectors comprising a first nucleic acid sequence encoding an antibody heavy chain and a second nucleic acid sequence encoding an antibody light chain. In some embodiments, the vector comprises a first nucleic acid sequence encoding SEQ ID NO: 9, 18, 25, or 35 and a second nucleic acid sequence encoding SEQ ID NO: 10, 19, 28 or 36.

In some embodiments, the vector comprises a first nucleic acid sequence encoding SEQ ID NO: 9 and a second nucleic acid sequence encoding SEQ ID NO: 10. In some embodiments, the vector comprises a first nucleic acid sequence encoding SEQ ID NO: 18 and a second nucleic acid sequence encoding SEQ ID NO: 19. In some embodiments, the vector comprises a first nucleic acid sequence encoding SEQ ID NO: 25 and a second nucleic acid sequence encoding SEQ ID NO: 10. In some embodiments, the vector comprises a first nucleic acid sequence encoding SEQ ID NO: 25 and a second nucleic acid sequence encoding SEQ ID NO: 19. In some embodiments, the vector comprises a first nucleic acid sequence encoding SEQ ID NO: 25 and a second nucleic acid sequence encoding SEQ ID NO: 28. In some embodiments, the vector comprises a first nucleic acid sequence encoding SEQ ID NO: 35 and a second nucleic acid sequence encoding SEQ ID NO: 36.

Also provided herein are compositions comprising a first vector comprising a nucleic acid sequence encoding an antibody heavy chain, and a second vector comprising a nucleic acid sequence encoding an antibody light chain. In some embodiments, the composition comprises a first vector comprising a nucleic acid sequence encoding SEQ ID NO: 9, 18, 25, or 35 and a second nucleic acid sequence encoding SEQ ID NO: 10, 19, 28 or 36.

In some embodiments, the composition comprises a first vector comprising a nucleic acid sequence encoding SEQ ID NO: 9 and a second vector comprising a nucleic acid sequence encoding SEQ ID NO: 10. In some embodiments, the composition comprises a first vector comprising a nucleic acid sequence encoding SEQ ID NO: 18 and a second vector comprising a nucleic acid sequence encoding SEQ ID NO: 19. In some embodiments, the composition comprises a first vector comprising a nucleic acid sequence encoding SEQ ID NO: 25 and a second vector comprising a nucleic acid sequence encoding SEQ ID NO: 10. In some embodiments, the composition comprises a first vector comprising a nucleic acid sequence encoding SEQ ID NO: 25 and a second vector comprising a nucleic acid sequence encoding SEQ ID NO: 19. In some embodiments, the composition comprises a first vector comprising a nucleic acid sequence encoding SEQ ID NO: 25 and a second vector comprising a nucleic acid sequence encoding SEQ ID NO: 28. In some embodiments, the composition comprises a first vector comprising a nucleic acid sequence encoding SEQ ID NO: 35 and a second vector comprising a nucleic acid sequence encoding SEQ ID NO: 36.

Nucleic acids of the present disclosure may be expressed in a host cell, for example, after the nucleic acids have been operably linked to an expression control sequence. Expression control sequences capable of expression of nucleic acids to which they are operably linked are well known in the art. An expression vector may include a sequence that encodes one or more signal peptides that facilitate secretion of the polypeptide(s) from a host cell. Expression vectors containing a nucleic acid of interest (e.g., a nucleic acid encoding a heavy chain or light chain of an antibody) may be transferred into a host cell by well-known methods, e.g., stable or transient transfection, transformation, transduction or infection. Additionally, expression vectors may contain one or more selection markers, e.g., tetracycline, neomycin, and dihydrofolate reductase, to aide in detection of host cells transformed with the desired nucleic acid sequences.

In another aspect, provided herein are cells, e.g., host cells, comprising the nucleic acids, vectors, or nucleic acid compositions described herein. A host cell may be a cell stably or transiently transfected, transformed, transduced or infected with one or more expression vectors expressing all or a portion of an antibody described herein. In some embodiments, a host cell may be stably or transiently transfected, transformed, transduced or infected with an expression vector expressing HC and LC polypeptides of an antibody of the present disclosure. In some embodiments, a host cell may be stably or transiently transfected, transformed, transduced, or infected with a first vector expressing HC polypeptides and a second vector expressing LC polypeptides of an antibody described herein. Such host cells, e.g., mammalian host cells, can express the antibodies that bind human TNFα as described herein. Mammalian host cells known to be capable of expressing antibodies include CHO cells, HEK293 cells, COS cells, and NS0 cells.

In some embodiments, the cell, e.g., host cell, comprises a vector comprising a first nucleic acid sequence encoding SEQ ID NO: 9, 18, 25, or 35 and a second nucleic acid sequence encoding SEQ ID NO: 10, 19, 28, or 36.

In some embodiments, the cell, e.g., host cell, comprises a first vector comprising a nucleic acid sequence encoding SEQ ID NO: 9, 18, 25, or 35 and a second vector comprising a nucleic acid sequence encoding SEQ ID NO: 10, 19, 28, or 36.

The present disclosure further provides a process for producing an antibody that binds human TNFα described herein by culturing the host cell described above, e.g., a mammalian host cell, under conditions such that the antibody is expressed and recovering the expressed antibody from the culture medium. The culture medium, into which an antibody has been secreted, may be purified by conventional techniques. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, N.Y. (1994).

The present disclosure further provides antibodies or antigen binding fragments thereof produced by any of the processes described herein.

In another aspect, provided herein are pharmaceutical compositions comprising an antibody, nucleic acid, or vector described herein. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 22nd ed. (2012), A. Loyd et al., Pharmaceutical Press).

The antibodies that bind human TNFα, nucleic acids, vectors, or pharmaceutical compositions described herein can be used for treating a TNFα associated disorder such as chronic autoinflammatory immune disorders, including but not limited to Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis, or Behcet's Disease

In some embodiments, provided herein are methods of treating a TNFα associated disorder, e.g., a chronic autoinflammatory immune disorder, in a subject (e.g., a human patient) in need thereof, comprising administering to the subject a therapeutically effective amount of an antibody that binds human TNFα, a nucleic acid encoding such an antibody that binds human TNFα, a vector comprising such a nucleic acid, or a pharmaceutical composition comprising such an antibody that binds human TNFα, nucleic acid or vector, as described herein. The antibodies, nucleic acids, vectors, or pharmaceutical compositions described herein may be administered by parenteral routes (e.g., subcutaneous, and intravenous). In embodiments, the TNFα associated disorder is a chronic autoinflammatory immune disorder. Such chronic autoinflammatory immune disorders include, but are not limited to, Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis or Behcet's Disease. In some embodiments, the subject being administered the therapeutically effective amount of the antibody that binds human TNFα received prior treatment with other anti-TNFα therapeutic, and wherein the subject developed anti-drug antibodies against the other anti-TNFα therapeutic. In such embodiments, the other anti-TNFα therapeutic is selected from Adalimumab, Infliximab, Golimumab, Certolizumab, or Etanercept. In yet further embodiments, the anti-human TNFα antibody as disclosed herein has low to no binding to anti-drug antibodies against at least four or more of other anti-TNFα therapeutic selected from the group consisting of Adalimumab, Infliximab, Golimumab, Certolizumab, and Etanercept. In yet further embodiments, the anti-human TNFα antibody as disclosed herein has low to no binding to anti-drug antibodies against at least three or more of other anti-TNFα therapeutic selected from the group consisting of Adalimumab, Infliximab, Golimumab, Certolizumab, and Etanercept. In yet further embodiments, the anti-human TNFα antibody as disclosed herein has low to no binding to anti-drug antibodies against at least two or more of other anti-TNFα therapeutic selected from the group consisting of Adalimumab, Infliximab, Golimumab, Certolizumab, and Etanercept. In yet other embodiments, the anti-human TNFα antibody as disclosed herein has low to no binding to anti-drug antibodies against Adalimumab.

Also provided herein are, antibodies that bind human TNFα, nucleic acids, vectors, or pharmaceutical compositions described herein for use in therapy. Furthermore, the present disclosure also provides, antibodies that bind human TNFα, nucleic acids, vectors, or pharmaceutical compositions described herein for use in the treatment of a TNFα associated disorder, e.g., chronic autoinflammatory immune disorders. Such chronic autoinflammatory immune disorders include, but are not limited to, Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis, and Behcet's Disease. The antibodies, nucleic acids, vectors, or pharmaceutical compositions described herein may be administered by parenteral routes (e.g., subcutaneous, and intravenous). In some embodiments of the present disclosure, the subject being administered the therapeutically effective amount of the antibody that binds human TNFα received prior treatment with other anti-TNFα therapeutic, and wherein the subject developed anti-drug antibodies against the other anti-TNFα therapeutic. In such embodiments, the other anti-TNFα therapeutic is selected from Adalimumab, Infliximab, Golimumab, Certolizumab, or Etanercept. In yet further embodiments, the anti-human TNFα antibody as disclosed herein has low to no binding to anti-drug antibodies against at least four or more of other anti-TNFα therapeutic selected from the group consisting of Adalimumab, Infliximab, Golimumab, Certolizumab, and Etanercept. In yet further embodiments, the anti-human TNFα antibody as disclosed herein has low to no binding to anti-drug antibodies against at least three or more of other anti-TNFα therapeutic selected from the group consisting of Adalimumab, Infliximab, Golimumab, Certolizumab, and Etanercept. In yet further embodiments, the anti-human TNFα antibody as disclosed herein has low to no binding to anti-drug antibodies against at least two or more of other anti-TNFα therapeutic selected from the group consisting of Adalimumab, Infliximab, Golimumab, Certolizumab, and Etanercept. In yet other embodiments, the anti-human TNFα antibody as disclosed herein has low to no binding to anti-drug antibodies against Adalimumab.

Provided herein are use of the antibodies that bind human TNFα, nucleic acids, vectors, or pharmaceutical compositions described herein in the manufacture of a medicament for the treatment of an TNFα associated disorder, e.g., a chronic autoinflammatory immune disorder. Such chronic autoinflammatory immune disorders include, but are not limited to, Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis, and Behcet's Disease.

In some embodiments, the antibody of the present disclosure binds human TNFα and has low to no binding to anti-drug antibodies against other anti-TNFα therapeutic. In such embodiments, the anti-human TNFα antibody of the present disclosure binds human TNFα, neutralizes soluble and membrane human TNFα, and inhibits TNF receptor mediated responses. In some embodiments the other anti-TNFα therapeutic is selected from Adalimumab, Infliximab, Golimumab, Certolizumab, or Etanercept. In some embodiments, the anti-human TNFα antibody of the present disclosure has low to no binding to anti-drug antibodies against at least four or more of other anti-TNFα therapeutic consisting of Adalimumab, Infliximab, Golimumab, Certolizumab, and Etanercept. In other embodiments, the anti-human TNFα antibody of the present disclosure has low to no binding to anti-drug antibodies against at least three or more of other anti-TNFα therapeutic consisting of Adalimumab, Infliximab, Golimumab, Certolizumab, and Etanercept. In other embodiments, the anti-human TNFα antibody of the present disclosure has low to no binding to anti-drug antibodies against at least two or more of other anti-TNFα therapeutic consisting of Adalimumab, Infliximab, Golimumab, Certolizumab, and Etanercept. In other embodiments, the anti-human TNFα antibody of the present disclosure has low to no binding to anti-drug antibodies against Adalimumab. In such embodiments, the anti-human TNFα antibody of the present disclosure is an IgG1. In further embodiments, the anti-human TNFα antibody of the present disclosure binds human TNFα and has low to no binding to anti-drug antibodies against other anti-TNFα therapeutic, wherein the anti-human TNFα antibody comprises a heavy chain (HC) and a light chain (LC), wherein the HC comprises SEQ ID NO: 9, 18, 25, or 35 and the LC comprises SEQ ID NO: 10, 19, 28, or 36. In such embodiments, the anti-human TNFα antibody of the present disclosure neutralizes human TNFα, and inhibits TNF receptor mediated responses. In further embodiments, the anti-human TNFα antibody of the present disclosure is an internalizing antibody. In yet further embodiments, the anti-human TNFα antibody of the present disclosure has low immunogenicity.

The term “TNFα” as used herein, unless stated otherwise, refers to soluble and/or membrane TNFα, and any native, mature TNFα that results from processing of a TNFα precursor protein in a cell. The term includes TNFα from any vertebrate source, including mammals such as canines, primates (e.g., humans and cynomolgus or rhesus monkeys) and rodents (e.g., mice and rats), unless otherwise indicated. The term also includes naturally occurring variants of TNFα, e.g., splice variants or allelic variants. The amino acid sequence of an example of human TNFα is known in the art, e.g., NCBI accession number: NP 000585 (SEQ ID NO: 39). The amino acid sequence of an example of cynomolgus monkey TNFα is also known in the art, e.g., UniProt reference sequence P79337 (SEQ ID NO: 45). The amino acid sequence of an example of rhesus macaque monkey TNFα is also known in the art, e.g., UniProt reference sequence P48094 (SEQ ID NO: 40). The amino acid sequence of an example of canine TNFα is also known in the art, e.g., GenBank accession number: CAA64403 (SEQ ID NO: 44). The term human “TNFα” is used herein to refer collectively to all known human TNFα isoforms and polymorphic forms. Sequence numbering used herein is based on the mature protein without the signal peptide.

The term “TNFR” or “TNF Receptors” as used herein, unless stated otherwise, refers to any native, mature TNFR e.g., TNFR1 (also known as p55 or p60) or TNFR2 (also known as p75 or p80). The term includes TNFR from any vertebrate source, including mammals such as canines, primates (e.g., humans and cynomolgus or rhesus monkeys) and rodents (e.g., mice and rats), unless otherwise indicated. The term also includes naturally occurring variants of TNFR, e.g., splice variants or allelic variants. The amino acid sequence of an example of human TNFR1 is known in the art, e.g., GenBank accession number: AAA61201 (SEQ ID NO: 48). The amino acid sequence of an example of human TNFR2 is known in the art, e.g., NCBI accession number: NP_001057 (SEQ ID NO: 49). The term “TNFR” is used herein to refer collectively to all known human TNFR isoforms and polymorphic forms.

The term “TNFα associated disorder” as used herein refers to a disorder associated with dysregulation of TNFα induced TNF receptor mediated signaling, such as disorders associated with dysregulation of TNFα induced TNFR1 and/or TNFR2 signaling. Such TNFα mediated disorders may for example include chronic autoinflammatory immune disorders, as disclosed herein.

The term “anti-drug antibodies” or “ADA” as used herein refers to antibodies formed in a mammal from an immune response to a therapeutic administered to that mammal. In some embodiments of the present disclosure, anti-drug antibodies formed against a therapeutic may neutralize the effects of that therapeutic, thus altering the therapeutic's pharmacokinetic (PK) and/or pharmacodynamic (PD) properties, interfering with the effect of the therapeutic, and/or reducing the efficacy, and/or diminishing clinical response to the therapeutic. Anti-drug antibodies to a therapeutic may also lead to adverse immune reaction in a patient such that the patient may not be a candidate for further treatment with that therapeutic. Examples of adverse immune reactions include but are not limited to infusion related reactions characterized by symptoms such as fever, pruritus, bronchospasms, or cardiovascular collapse during or within the first day after drug administration (Atiqi, S., Front Immunol., 2020, 26(11): 312).

The term “low to no binding” to anti-drug antibodies as used herein refers to the binding of the anti-human TNFα antibodies of the present disclosure to anti-drug antibodies against other anti-TNFα therapeutic wherein such binding is determined to be below the cut-off point of the assay used to measure the binding or is within the pre-determined variability range of the assay. In such methods, the cut-off point is a pre-determined threshold that is used to identify positive binding to anti-drug antibodies. In some embodiments the pre-determined variability of the assay is less than about 20% above the cut-off point of the assay. In such embodiments, binding of the anti-human TNFα antibodies of the present disclosure to anti-drug antibodies against other therapeutic (e.g., Adalimumab) that is less than about 20% above the cut-off point of the assay is considered low binding. In some embodiments, binding of the anti-human TNFα antibodies of the present disclosure to anti-drug antibodies against other therapeutic (e.g., Adalimumab) that is at or below the cut-off point of the assay is considered no binding.

The term “other anti-TNFα therapeutic” refers to an agent which binds TNFα and inhibits TNF receptor mediated responses, not including the anti-human TNFα antibodies described herein. Such an agent may include, but is not limited to, an antibody, antibody fragment or antigen binding fragment, which comprise at least a portion of an antibody retaining the ability to interact with an antigen such as Fab, Fab′, F(ab′)2, Fv fragments, scFv, scFab, disulfide-linked Fvs (sdFv), a Fd fragment or linear antibodies, which may be for example, fused to an Fc region or an IgG heavy chain constant region. In some embodiments, the other anti-TNFα therapeutic may be for example, Adalimumab, Infliximab, Golimumab, Certolizumab, and/or Etanercept.

The term “antibody” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4). Embodiments of the present disclosure also include antibody fragments or antigen binding fragments, the term “antibody fragments or antigen binding fragments” comprise at least a portion of an antibody retaining the ability to interact with an antigen such as for example, Fab, Fab′, F(ab′)2, Fv fragments, scFv, scFab, disulfide-linked Fvs (sdFv), a Fd fragment or linear antibodies, which may be for example, fused to an Fc region or an IgG heavy chain constant region.

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. The heavy chain constant region refers to a region of an antibody, which comprises the Fc region and CH1 domain of the antibody heavy chain. Each light chain is comprised of a light chain variable region (VL) and a light chain constant region. The IgG isotype may be further divided into subclasses (e.g., IgG1, IgG2, IgG3, and IgG4). The numbering of the amino acid residues in the constant region is based on the EU index as in Kabat. Kabat et al, Sequences of Proteins of Immunological Interest, 5th edition, Bethesda, Md.: U.S. Dept. of Health and Human Services, Public Health Service, National Institutes of Health (1991). The term EU Index numbering or EU numbering is used interchangeably herein.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). A combination of IMGT and North CDR definitions were used for the exemplified anti-human TNFα antibodies as described herein.

The term “Fc region” as used herein, refers to a region of an antibody, which comprises the CH2 and CH3 domains of the antibody heavy chain. Optionally, the Fc region may include a portion of the hinge region or the entire hinge region of the antibody heavy chain. Biological activities such as effector function are attributable to the Fc region, which vary with the antibody isotype. Examples of antibody effector functions include, Fc receptor binding, antibody-dependent cell mediated cytotoxicity (ADCC), antibody-dependent cell mediated phagocytosis (ADCP), C1q binding, complement dependent cytotoxicity (CDC), phagocytosis, down regulation of cell surface receptors (e.g. B cell receptor); and B cell activation.

The term “epitope” as used herein, refers to the amino acid residues of an antigen, that are bound by an antibody. An epitope can be a linear epitope, a conformational epitope, or a hybrid epitope. The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody. The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody. An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018; Holst et al., Molecular Pharmacology 1998, 53(1): 166-175), including but not limited to, X-ray crystallography, nuclear magnetic resonance (NMR) spectroscopy, site-directed mutagenesis, species swap mutagenesis, alanine-scanning mutagenesis, steric hindrance mutagenesis, hydrogen-deuterium exchange (HDX), and cross-blocking assays.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

The terms “nucleic acid” as used herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

The term “subject” as used herein, refers to a mammal, including, but are not limited to, a human, chimpanzee, ape, monkey, cattle, horse, sheep, goat, swine, rabbit, dog, cat, rat, mouse, guinea pig, and the like. Preferably, the subject is a human.

The term “therapeutically effective amount”, as used herein, refers to an amount of a protein or nucleic acid or vector or composition that will elicit the desired biological or medical response of a subject, for example, reduction or inhibition of a protein activity, or ameliorate symptoms, alleviate conditions, slow or delay disease progression, or prevent a disease, etc. In a non-limiting embodiment, the term “a therapeutically effective amount” refers to the amount necessary (at dosages and for periods of time and for the means of administration) of a protein or nucleic acid or vector or composition that, when administered to a subject, is effective to at least partially alleviate, inhibit, prevent and/or ameliorate a condition, or a disorder or a disease to achieve the desired therapeutic result. A therapeutically effective amount of the protein or nucleic acid or vector or composition may vary according to factors such as the disease state, age, sex, and weight of the individual, and the ability of the protein or nucleic acid or vector or composition to elicit a desired response in the individual. A therapeutically effective amount is also one in which any toxic or detrimental effects of the protein or nucleic acid or vector or composition of the present disclosure are outweighed by the therapeutically beneficial effects.

The term “inhibits” as used herein, refers to for example, a reduction, lowering, slowing, decreasing, stopping, disrupting, abrogating, antagonizing, or blocking of a biological response or activity, but does not necessarily indicate a total elimination of a biological response.

The term “treatment” or “treating” as used herein, refers to all processes wherein there may be a slowing, controlling, delaying or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The term “neutralize”, as used herein, refers to the ability of an antibody, antibody fragment or a binding molecule to counteract or render inactive or ineffective at least one activity or function of an antigen.

The term “about” as used herein, means within 10%.

As used herein, the term “a”, “an”, “the”, and similar terms used in the context of the present disclosure (especially in the context of the claims) are to be construed to cover both the singular and plural unless otherwise indicated herein or clearly contradicted by the context.

### BRIEF DESCRIPTION OF THE DRAWINGS

FIG. 1 shows that exemplified anti-human TNFα antibody Ab6 has low to no binding to anti-drug antibodies against Adalimumab formed in cynomolgus monkeys hyperimmunized with Adalimumab.

FIG. 2 shows that exemplified anti-human TNFα antibody Ab6 has low to no binding to anti-drug antibodies against Adalimumab formed in human patients treated with Adalimumab.

### EXAMPLES

The following examples are offered to illustrate, but not to limit, the claimed invention.

### Example 1: Generation and Engineering of Anti-Human TNFα Antibodies

Antibody generation: To develop antibodies specific to human TNFα, transgenic mice with human immunoglobulin variable regions were immunized with recombinant human TNFα. Screening was done with human TNFα and the cross reactivity with other TNFα species was tested. Antibodies cross reactive to cynomolgus monkey were cloned, expressed, and purified by standard procedures, and tested for neutralization in a TNFα induced cytotoxicity assay. Antibodies were selected and engineered in their CDRs, variable domain framework regions, and IgG isotype to improve binding affinities and developability properties such as, stability, solubility, viscosity, hydrophobicity, and aggregation.

The amino acid sequence of human TNFα is provided as SEQ ID NO: 39, the amino acid sequence of cynomolgus monkey TNFα is provided as SEQ ID NO: 45.

The TNFα antibodies can be synthesized and purified by well-known methods. An appropriate host cell, such as Chinese hamster ovarian cells (CHO), can be either transiently or stably transfected with an expression system for secreting antibodies using a predetermined HC:LC vector ratio if two vectors are used, or a single vector system encoding both heavy chain and light chain. Clarified media, into which the antibody has been secreted, can be purified using the commonly used techniques.

Antibody engineering to improve viscosity: The parental TNFα antibody lineage was found to have high viscosity upon concentration. Viscosity is a key developability criteria for assessing feasibility of delivery of a therapeutic antibody via autoinjector. Mutagenesis analysis of the antibody required a fine balancing of improving biophysical properties and retaining desirable affinity and potency without increasing immunogenicity risk. In-silico modeling of the parental antibody was used to identify regions of charge imbalance in the surface comprised of the 6 complementary determining regions (CDRs). The antibodies generated from the mutagenesis were screened for TNFα binding, and those antibodies which retained or improved target binding as compared to the parental mAb (as determined by ELISA) and had desirable viscosity and other developability properties were selected for further development.
Antibody engineering to reduce the risk of immunogenicity: The exemplified anti-human TNFα antibodies were further tested in an MHC-associated peptide proteomics (MAPPS) assay to determine immunogenicity risk. Briefly, major histocompatibility complex (MHC) bound peptides were identified for antibodies with specific CDR sequences. A CDR library having mutations identified as potentially reducing immunogenicity was constructed and screened for TNFα binding. Antibodies were screened and selected to optimize by engineering for low immunogenicity risk whilst balancing maintaining desirable binding affinity to TNFα and other desirable developability properties.

Tables 1 and 2 show the exemplified anti-human TNFα antibody sequences engineered to balance reduced viscosity, low immunogenicity risk and other desirable developability properties while retaining desirable binding affinity to human TNFα.

TABLE 1
CDR amino acid sequences of exemplified
anti-human TNFa antibodies

| TNFα | CDR Sequence | | | | | |
|---|---|---|---|---|---|---|
| Antibody | HCDR1 | HCDR2 | HCDR3 | LCDR1 | LCDR2 | LCDR3 |
| Ab1 | SEQ ID | SEQ ID | SEQ ID | SEQ ID | SEQ ID | SEQ ID |
| | NO: 1 | NO: 2 | NO: 3 | NO: 4 | NO: 5 | NO: 6 |
| Ab2 | SEQ ID | SEQ ID | SEQ ID | SEQ ID | SEQ ID | SEQ ID |
| | NO: 1 | NO: 2 | NO: 13 | NO: 14 | NO: 5 | NO: 15 |
| Ab3 | SEQ ID | SEQ ID | SEQ ID | SEQ ID | SEQ ID | SEQ ID |
| | NO: 22 | NO: 23 | NO: 13 | NO: 4 | NO: 5 | NO: 6 |
| Ab4 | SEQ ID | SEQ ID | SEQ ID | SEQ ID | SEQ ID | SEQ ID |
| | NO: 22 | NO: 23 | NO: 13 | NO: 14 | NO: 5 | NO: 15 |
| Ab5 | SEQ ID | SEQ ID | SEQ ID | SEQ ID | SEQ ID | SEQ ID |
| | NO: 22 | NO: 23 | NO: 13 | NO: 14 | NO: 5 | NO: 6 |
| Ab6 | SEQ ID | SEQ ID | SEQ ID | SEQ ID | SEQ ID | SEQ ID |
| | NO: 1 | NO: 2 | NO: 30 | NO: 31 | NO: 5 | NO: 32 |

TABLE 2
Amino Acid sequences of exemplified
anti-human TNFα antibodies

| TNFα | | | | |
|---|---|---|---|---|
| Antibody | VH | VL | HC | LC |
| Ab1 | SEQ ID | SEQ ID | SEQ ID | SEQ ID |
| | NO: 7 | NO: 8 | NO: 9 | NO: 10 |
| Ab2 | SEQ ID | SEQ ID | SEQ ID | SEQ ID |
| | NO: 16 | NO: 17 | NO: 18 | NO: 19 |
| Ab3 | SEQ ID | SEQ ID | SEQ ID | SEQ ID |
| | NO: 24 | NO: 8 | NO: 25 | NO: 10 |
| Ab4 | SEQ ID | SEQ ID | SEQ ID | SEQ ID |
| | NO: 24 | NO: 17 | NO: 25 | NO: 19 |
| Ab5 | SEQ ID | SEQ ID | SEQ ID | SEQ ID |
| | NO: 24 | NO: 27 | NO: 25 | NO: 28 |
| Ab6 | SEQ ID | SEQ ID | SEQ ID | SEQ ID |
| | NO: 33 | NO: 34 | NO: 35 | NO: 36 |

### Example 2. Binding Affinity of Exemplified Anti-Human TNFα Antibodies

Binding Affinity, method 1: Binding affinities of the antibodies to human, rhesus macaque, mouse, rat, rabbit, and canine TNFα protein were tested in an antigen-down ELISA format. Briefly, 384-well high binding plates (Greiner Bio-one #781061) were coated with 20 μL per well of 1 μg/mL of human TNFα (Syngene), 2 μg/mL of rhesus macaque TNFα (R&D Systems, Cat #1070-RM), 2 μg/mL of mouse TNFα (R&D Systems, Cat #410-MT/CF), or 2 μg/mL of rat TNFα (R&D Systems, Cat #510-RT-CF), 2 μg/mL of rabbit TNFα (R&D Systems, Cat #5670-TG/CF), or 2 μg/mL of canine TNFα (R&D Systems, Cat #1507-CT/CF) diluted in carbonate buffer pH 9.3 (0.015 M Na₂CO₃ and 0.035 M NaHCO₃) and stored at 4° C. overnight. Next day, the plates were blocked with 80 μL casein (Thermo Fisher Pierce, Cat #37528) for 1 h at room temperature, blocking buffer was removed, and 20 μL of titrated purified antibody expressed in CHO cells (starting concentration at 20 μg/mL diluted in casein and titrated 3-fold, 8 points down) was added to the plate. The plate was incubated at 37° C. for 90 min, then washed three times in PBS/0.1% Tween. 20 μL of secondary antibody reagent goat-anti-human-kappa-AP (Southern Biotech, Cat #2060-04) with 1:1500 dilution was added to the plate and incubated for 45 minutes at 37° C. Plates were washed 3 times in PBS/0.1% Tween, and 20 μL of alkaline phosphatase substrate solution diluted to 1:35 in molecular grade water was added to every well. Once the color developed (approximately 15-30 min), plates were read at 560 nM OD on a Molecular Device Spectramax plate reader and data was acquired using Softmax Pro 4.7 software. Data analysis was performed in GraphPad Prism.

The results as demonstrated in Table 3 show that the exemplified anti-human TNFα antibodies Ab1, Ab2, Ab3, Ab4, Ab5, and Ab6 bound human, rhesus macaque monkey, and canine TNFα with desirable affinities.

TABLE 3
Binding affinity of exemplified anti-human TNFα antibodies
to human, rhesus macaque, and canine TNFα

| | Human TNFα | Rhesus Macaque TNFα | Canine TNFα |
|---|---|---|---|
| Antibody | EC₅₀ (μg/mL) | EC₅₀ (μg/mL) | EC₅₀ (μg/mL) |
| Ab1 | 0.2225 | 0.1555 | 0.2204 |
| Ab2 | 0.222 | 0.1621 | 0.24 |
| Ab3 | 0.2164 | 0.1414 | 0.2401 |
| Ab4 | 0.2134 | 0.1444 | 0.2306 |
| Ab5 | 0.2201 | 0.1607 | 0.1757 |
| Ab6 | 0.1313 | 0.1148 | 0.162 |

Binding affinity, method 2: An MSD Sector S 600 instrument (Meso Scale Discovery, Rockville, Md.) was used for reading MSD plates. Human and cynomolgus monkey TNFα were biotinylated using a Thermo-Fisher biotinylation kit. MSD assay plates were prepared as follows: a multi-array streptavidin-coated 96-well plate (Meso Scale Discovery, Cat #L15SA-1) was coated for one hour at room temperature (approximately 25° C. with 40 μL per well of 1 μg/mL solution of either biotinylated human TNFα or biotinylated cynomolgus monkey TNFα in PBS. The plates were washed 3 times in PBS +0.1% Tween (PBST) following coating, then blocked for 1 hour at room temperature with 1% bovine serum albumin (BSA) in PBS. The plates were then washed 3× with PBS prior to adding sample solutions. Solution equilibrium titration (SET) samples were prepared in 1% BSA. Anti-human TNFα antibody Ab1 was diluted to 10 μM and TNFα was serially diluted for a total of 12 dilutions. TNFα titrations and fixed antibody solutions were combined 1:1 to prepare the SET solutions. SET solutions were incubated at 37° C. for approximately 72 hours to allow binding to reach equilibrium. 40 μL of the SET solutions was transferred to the prepared MSD plate in triplicate rows and incubated at room temperature for 2.5 minutes to capture free antibody with agitation by manually tapping the plate lightly. Following incubation, the plate was washed 3 times with PBST. Then, 40 μL of 1 μg/mL SULFO-Tag anti-human/NHP kappa antibody (Meso Discovery Scale, Cat #D20TF-6) in 1% BSA was added to all wells. This was incubated statically at room temperature for one hour. The plate was then washed 3 times with PB ST, then 150 μL of MSD GOLD Read Buffer A (Meso Scale Discovery, Cat #R92TG-2) was added before reading the plate. Dilution series were prepared in triplicate in each individual experiment, and the three independent replicate experiments were conducted. The K_{D} was determined using a quadratic kinetic model in XLfit from the MSD-SET data. Replicate K_{D} values were entered into GraphPad Prism for both human and cynomolgus monkey TNFα to determine standard deviation statistics.

The results of this assay show that the exemplified anti-human TNFα antibody Ab1 binds to human TNFα with a K_{D} of 8.5±1.6 μM and to cynomolgus monkey TNFα with a K_{D} of 21.2±7.4 μM.

### Example 3: Functional Activities of Exemplified Anti-Human TNFα Antibodies

Internalization of membrane bound TNFα antibodies: Internalization of exemplified anti-human TNFα antibodies upon binding to membrane expressed TNFα was tested in vitro on a CHO cell line stably transfected to express membrane human TNFα (non-cleavable TNFα). Briefly, F(ab′)2 fragment goat anti-human IgG (Jackson #109-006-098) was conjugated to pHrodo pH-sensitive dye (Fisher P36014) using manufacturer's protocol. The exemplified anti-human TNFα antibodies were incubated with equimolar amount of F(ab′)2 goat anti-hIgG-pHrodo in CHO growth media for 30 min room temp. The antibody-dye mixtures were added to CHO human TNFα transfected cells, then incubated in a 5% CO₂ shaker incubator at 37° C. for 3, 6, and 24 hour time points. Final concentrations of the exemplified TNFα antibodies were 10 μg/mL and 3.3 μg/mL. Cells were washed at indicated timepoints and analyzed on a BD Fortessa flow cytometer.

The results as demonstrated in Table 4, show that the anti-human TNFα antibodies tested, were internalized into the CHO cells upon binding to membrane human TNFα expressed on CHO cells at 3.33 μg/mL and 10 μg/mL. The results further showed that the anti-human TNFα antibodies were internalized into the lysosome upon binding membrane TNFα expressed on the lysosome (data not shown).

TABLE 4
Internalization of exemplified anti-human TNFα antibodies

| | | Internalization | Internalization | Internalization |
|---|---|---|---|---|
| | Antibody | at 3 hr | at 6 hr | at 24 hr |
| Antibody | μg/mL | (Phrodo MFI) | (Phrodo MFI) | (Phrodo MFI) |
| Ab1 | 3.33 | 1913 | 3663 | 1152 |
| Ab2 | 3.33 | 1811 | 3266 | 1168 |
| Ab3 | 3.33 | 2107 | 3823 | 1197 |
| Ab4 | 3.33 | 2128 | 3518 | 1177 |
| hIgG1 | 3.33 | 304 | 318 | 313 |
| Isotype | | | | |
| Ab1 | 10.0 | 3083 | 5989 | 4575 |
| Ab2 | 10.0 | 2686 | 5158 | 4643 |
| Ab3 | 10.0 | 3339 | 6281 | 4811 |
| Ab4 | 10.0 | 2998 | 5750 | 4352 |
| hIgG1 | 10.0 | 339 | 340 | 337 |
| Isotype | | | | |

Inhibition of soluble and membrane TNFα induced cell killing: Inhibition of soluble and membrane TNFα induced cell killing by the exemplified anti-human TNFα antibodies was evaluated in an in vitro cell based assay using L929 mouse fibrosarcoma cells which naturally express the TNF receptor. When combined with Actinomycin-D, TNFα induces classical apoptosis in these cells, resulting in rapid cell death due to excessive formation of reactive oxygen intermediates which can be rescued by TNFα neutralization. The quantity of viable cells can be measured using MTS-tetrazolium cytotoxicity assay, where the mitochondrial dehydrogenase enzymes in metabolically active cells reduces the MTS-tetrazolium into a colored formazan product, which can be detected with a microplate reader (Biotek Cytation 5 Imaging Multi-Mode Reader).

Inhibition of soluble TNFα induced cell killing: To evaluate the ability of the exemplified anti-human TNFα antibodies to inhibit soluble TNFα induced cell killing, L929 cells were treated with either human TNFα or cynomolgus monkey TNFα, separately. L929 cells resuspended at 10,000 cells/100 μL in assay medium (lx DMEM media, 10% FBS, 1% Pen-Strep, 1% MEM essential amino acids, 1% L-glutamine, 1% sodium pyruvate) were added to 96-well plates and placed in a tissue culture incubator overnight. The next day, exemplified antibodies were diluted at concentrations ranging from 15 μg/mL to 0.0005 μg/mL (with three-fold dilution), and 100 μL of each concentration of the exemplified anti-human TNFα antibodies was added in duplicate to wells containing one of the two following conditions: 200 pg/mL recombinant human TNFα, or 750 pg/mL recombinant cynomolgus TNFα, and plates were incubated for 30 min at room temperature. Human IgG1 isotype control antibody was used as a negative control. The antibody/TNFα/actinomycin-D mixture was then transferred to the 96-well plates with L929 adherent cells, and incubated in a tissue culture incubator for 18 hrs. The assay medium was removed, and 100 μL of MTS-tetrazolium substrate mixture was added to the wells and plates were incubated in a tissue culture incubator for 2 hrs. To determine cell death, plates were read at 490 nm on a microplate reader (Biotek Cytation 5 Imaging Multi-Mode Reader). Results were expressed as the concentration where 50% of the TNFα-induced cytotoxicity was inhibited (IC₅₀, average of two independent experiments ±SEM) by the exemplified anti-human TNFα antibodies, calculated using a 3-parameter sigmoidal fit of the data (GraphPad Prism 9). The IC₅₀ values are shown in Table 5a.

Inhibition of membrane TNFα induced cell killing: To evaluate the ability of the exemplified anti-human TNFα antibodies to inhibit membrane TNFα induced cell killing, the non-cleavable TNFα construct was stably transfected into Chinese hamster ovary (CHO) cells to generate cell surface (membrane) TNFα expressing CHO cells. The non-cleavable TNFα construct was generated with known mutations at the cleavage sites of the TNFα which allowed for expression of bioactive TNFα on the cell surface (Mueller et. al. 1999) in the absence of TNFα cleavage. Incubation of L929 cells with CHO cells expressing the human non-cleavable TNFα resulted in rapid L929 cell death. To determine whether the exemplified anti-human TNFα antibodies could neutralize the observed cell killing a dose range from 15 μg/mL to 0.0005 μg/mL (with three-fold dilution) was evaluated for each of the exemplified antibodies. Each concentration of the exemplified anti-human TNFα antibodies (100 μL/well) was added in duplicate to plates containing 500 CHO TNFα transfectant cells/well+6.25 μg/mL actinomycin-D. The antibody plus CHO cell mixtures were incubated for 30 min at room temperature and then added into the L929 cell plate. A human IgG1 isotype control antibody was used as a negative control and tested at similar dose range to the anti-human TNFα antibodies. The L929 cell death was determined essentially as described for the soluble TNFα induced cell killing assay. The IC₅₀ values are shown in Table 5b.

The results, as demonstrated in Table 5a and 5b, showed that the exemplified anti-human TNFα antibodies inhibited soluble human TNFα or soluble cynomolgus TNFα induced L929 cell killing, and human membrane TNFα induced cell killing of L929 cells. A dose dependent inhibition of cell killing response was observed. Specifically, the IC₅₀ for inhibition of soluble human TNFα induced cell killing (Table 5a) by the exemplified anti-human TNFα antibodies tested ranged from about 0.13 μg/mL to about 0.22 μg/mL, and from about 0.02 μg/mL to about 0.3 μg/mL for inhibition of soluble cynomolgus TNFα induced cell killing. The IC₅₀ for inhibition of human membrane TNFα induced cell killing (Table 5b) by the exemplified anti-human TNFα antibodies tested ranged from about 0.13 μg/mL to about 0.12 μg/mL. The negative control hIgG1 isotype as expected, did not inhibit TNFα induced cell killing.

TABLE 5a
Exemplified anti-human TNFα antibodies inhibit soluble human and
soluble cynomolgus monkey TNFα induced cell killing of L929 cells

| | Soluble | | Soluble cynomolgus | |
|---|---|---|---|---|
| | human TNFα | | monkey TNFα | |
| | IC₅₀ | Std. Error | IC₅₀ | Std. Error |
| Antibody | (μg/mL) | of Mean | (μg/mL) | of Mean |
| hIgG1 Isotype | n/a | n/a | n/a | n/a |
| Ab1 | 0.16 | 0.02 | 0.02 | 0.005 |
| Ab2 | 0.13 | 0.03 | 0.02 | 0.004 |
| Ab3 | 0.19 | 0.02 | 0.02 | 0.004 |
| Ab4 | 0.19 | 0.00 | 0.02 | 0.005 |
| Ab5 | 0.22 | 0.04 | 0.03 | 0.009 |

TABLE 5b
Exemplified anti-human TNFα antibodies inhibit membrane
TNFα-induced cell killing of L929 cells

| Antibody | IC₅₀ (μg/mL) | Std. Error of Mean |
|---|---|---|
| hIgG1 Isotype | n/a | n/a |
| Ab1 | 0.13 | 0.04 |
| Ab2 | 0.13 | 0.07 |
| Ab3 | 0.15 | 0.08 |
| Ab4 | 0.13 | 0.05 |
| Ab5 | 0.20 | 0.09 |

### Example 4: Characterization of Exemplified Anti-Human TNFα Antibodies Binding to Anti-Drug Antibodies Against Adalimumab

Binding to Cynomolgus monkey anti-drug antibodies against Adalimumab: Binding of exemplified anti-human TNFα antibodies to anti-drug antibodies against Adalimumab (anti-Adalimumab antibodies) obtained from affinity purified hyperimmune monkey serum (AP-HIMS) from cynomolgus monkeys hyperimmunized with Adalimumab was evaluated. An Adalimumab-AffiGe110 was used to purify the anti-Adalimumab antibodies from the Adalimumab hyperimmunized Cynomolgus monkeys. The anti-Adalimumab antibodies were detected using a titration of AP-HIMS in an ACE-Bridge assay. The assay was developed following the FDA Guidance on Immunogenicity testing. Briefly, streptavidin-coated 96-well plates (Pierce, 15500) were washed with 1×TBST (Boston BioProducts, IBB-181X), and coated with 30 nM biotinylated Adalimumab at 100 μL/well in TBST/0.1% bovine serum albumin (BSA; Sigma, A7888) for 1 h at room temperature. Plates were washed three times with TBST, and affinity purified anti-Adalimumab antibodies were diluted 1:10 with TBS (Fisher, BP2471-1), and added at 100 uL/well to the coated plates and incubated overnight at 4° C. The following day, plates were washed three times with TBST and the captured anti-Adalimumab antibodies were acid eluted using 65 μL/well of 300 mM acetic acid (Fisher Scientific, A38-500) for 5 min at room temperature. Polypropylene 96-well plates (Corning, 3359) were then loaded with 50 μL of 1 μg/mL each of biotinylated Adalimumab and ruthenium-labeled Adalimumab in neutralizing buffer (0.375 M Tris, 300 mM NaCl, pH 9). Next, 50 μL of the acid eluted samples were added to the polypropylene plate containing the mixture in neutralizing buffer and the ADA and were allowed to bridge to the labeled antibodies for 1 h at room temperature. MSD Gold 96-well streptavidin plates (Mesoscale, L15SA-1) were washed and blocked with TBS+1% BSA for 1 h at room temperature, then washed, and 80 μL of bridged samples were added to the plate for 1 h. The plates were washed three times with TBST, and 150 μL/well of 2×MSD Buffer (Mesoscale, R92TC-2) was added to the plates. Plates were read on an MSD SQ120 reader to provide the Tier 1 signal expressed as electro chemiluminescent units (ECLU).

The same AP-HIMS was also tested in the ACE-Bridge assay to detect antibodies against exemplified anti-human TNFα antibody Ab6, following essentially the same method outlined above for Adalimumab, but using biotin and ruthenium-labeled Ab6. The resulting ECLU signal was then plotted as a function of the concentration of AP-HEWS tested.

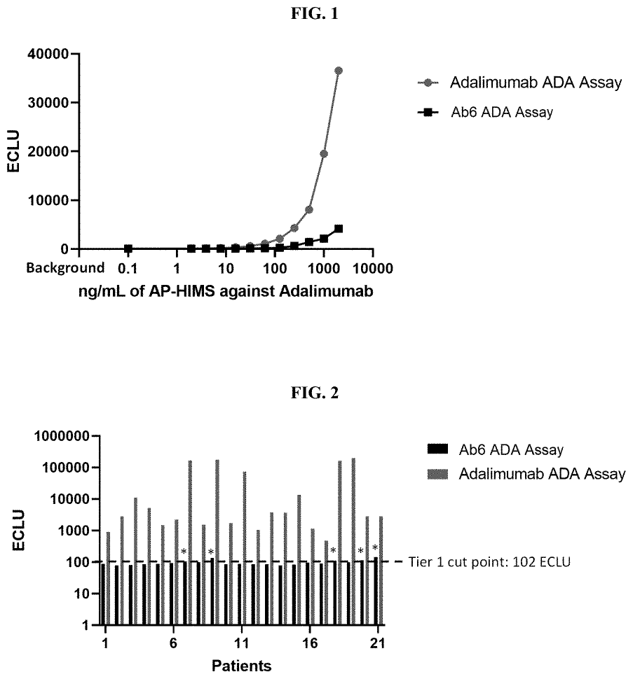

The results as demonstrated in FIG. 1, show that the exemplified anti-human TNFα antibody Ab6 had low to no binding to the anti-drug antibodies against Adalimumab (maximum ECLU signal of 4000) purified from serum from Cynomolgus monkey hyperimmunized with Adalimumab, when compared to binding of Adalimumab to its own anti-drug antibodies (maximum ECLU signal 40000). Specifically, the results showed that the exemplified anti-human TNFα antibody Ab6 only recognized about 10% of the anti-drug antibodies against Adalimumab raised by the cynomolgus monkeys suggesting that this binding is likely due to shared sequences located away from the CDR regions, such as the antibody constant region.

Binding to human patient anti-drug antibodies against Adalimumab: Binding of exemplified anti-human TNFα antibodies to anti-drug antibodies against Adalimumab (anti-Adalimumab antibodies) in 21 patient serum samples obtained from Adalimumab-treated patients enrolled in the study RA-BEAM was evaluated. The 21 serum samples were collected post-baseline, and were confirmed to have high ADA titers against Adalimumab, by using the methods essentially as described for the Cynomolgus monkey ADA evaluation. The 21 serum samples were then evaluated for binding to exemplified anti-human TNFα antibody Ab6 using the methods essentially as described for the Cynomolgus monkey ADA evaluation.

The results as demonstrated in FIG. 2, show that the exemplified anti-human TNFα antibody Ab6 had low to no binding to the anti-drug antibodies against Adalimumab in 16 of the 21 patient samples tested (ECLU signal was below the cut-off point of the assay (102 ECLU)). In determining immunogenicity, the cut-off point is a threshold that is used to identify “putative positive”, or anti-drug antibody containing, samples. As shown in FIG. 2, five out of 21 samples had an ECLU signal above the cut-off point, but were all less than 20% above the cut-off point, and therefore determined to be within the variability of the assay. The significantly low or no binding of anti-human TNFα antibody Ab6 to the anti-drug antibodies against Adalimumab in human patients treated with Adalimumab indicated that the Ab6 and Adalimumab antibody sequences are sufficiently different, such that, the ADA raised against Adalimumab by the human subjects tested, which are specific to epitopes present in Adalimumab, are not shared by Ab6, and thus not significantly recognized by Ab6. These results indicate potential use of the exemplified anti-human TNFα antibodies for treatment of patients who develop diminished clinical response or adverse reactions to anti-drug antibodies against other TNFα therapeutics such as Adalimumab.

### Example 5: Immunogenicity Assessment

DC internalization assay, MAPPS assay, and T cell proliferation assay on LCDR1 and HCDR3 peptide clusters were performed to evaluate immunogenicity risk of the exemplified TNFα antibodies.

Dendritic cell internalization assay: The ability of human CD14+ monocytes derived from dendritic cells (DC) to internalize the exemplified anti-human TNFα antibodies was assessed. CD14+ monocytes were isolated from periphery blood mononuclear cells (PBMCs), cultured and differentiated into immature dendritic cells (with IL-4 and GM-CSF), all using standard protocols. To obtain mature DCs, cells were treated with 1 μg/mL LPS for 4 hours.

Exemplified anti-human TNFα antibodies were diluted at 8 μg/mL in complete RPMI medium and mixed at equal volume with detection probe Fab-TAMRA-QSY7 diluted to 5.33 m/mL in complete RPMI medium, and incubated for 30 min at 4° C. in the dark for complex formation, then added to immature and mature DC cultures and incubated for 24 h at 37° C. in a CO₂ incubator. Cells were washed with 2% FBS PBS and resuspended in 100 μL 2% FBS PBS with Cytox Green live/dead dye. Data was collected on a BD LSR Fortessa X-20 and analyzed in FlowJo. Live single cells were gated, and percent of TAMRA fluorescence positive cells was recorded as the readout. To allow the comparison of molecules with data generated from different donors, a normalized internalization index was used. The internalization signal was normalized to IgG1 isotype (normalized internalization index=0) and an internal positive control PC (normalized internalization index=100) using Formula 1:

$$100 \times \frac{{X}_{T AMRA} - {I}_{g} G 1 {isotype}_{TAMRA}}{P {C}_{T A M R A} - {I}_{g} G 1 {isotype}_{T A M R A}} \qquad ( 1 )$$

where X_{TAMRA}, IgG1 isotype_{TAMRA}, and PC_{TAMRA} were the percent of TAMRA-positive population for the test molecule X, IgG1 isotype, and PC respectively.

The results as demonstrated in Table 6, show that the tested anti-human TNFα antibodies were internalized into the cell upon binding to TNFα on the immature and mature dendritic cells.

TABLE 6
DC internalization of exemplified anti-human TNFα antibodies

| | Immature Dendritic Cell | Mature Dendritic Cell |
|---|---|---|
| Antibody | Internalization Index | Internalization Index |
| Ab1 | 19.4 | 26.7 |
| Ab2 | 21.9 | 38.3 |
| Ab3 | 20.0 | 35.2 |
| Ab4 | 33.6 | 43.1 |
| Ab5 | 54.6 | 52.7 |

MHC-associated peptide proteomics (MAPPs) Assay: MAPPs profiles the MHC-II presented peptides on human dendritic cells previously treated with exemplified anti-human TNFα antibodies. CD14+ monocytes were isolated from periphery blood mononuclear cells (PBMCs), cultured and differentiated into immature dendritic cells (with IL-4 and GM-CSF) using standard protocols. Exemplified antibodies were added to the immature dendritic cells on day 4 and fresh media containing LPS to transform the cells into mature dendritic cells was exchanged after 5-hour incubation. The matured dendritic cells were lysed in RIPA buffer with protease inhibitors and DNAse the following day. Immunoprecipitation of MHC-II complexes was performed using biotinylated anti-MHC-II antibody coupled to streptavidin beads. The bound complex was eluted and filtered. The isolated MHC-II peptides were analyzed by a mass spectrometer. Peptide identifications were generated by an internal proteomics pipeline using search algorithms with no enzyme search parameter against a bovine/human database with test sequences appended to the database. Peptides identified from the exemplified antibodies were aligned against the parent sequence.

The results as demonstrated in Table 7, showed that the exemplified anti-human TNFα antibodies had varying degree of presentation by MAPPs. Ab1 demonstrated the lowest MAPPs presentation with 1 non-germline cluster in 3 of the 10 donors tested.

TABLE 7
MAPPs analysis of exemplified anti-human TNFα antibodies

| | Number of | Number of |
|---|---|---|
| | non-germline | donors containing |
| Samples | clusters across all donors | ≥1 cluster |
| Ab1 | 1 | 3/10 |
| Ab2 | 2 | 6/10 |
| Ab3 | 2 | 5/10 |
| Ab4 | 3 | 5/10 |
| Ab5 | 3 | 8/10 |

T cell proliferation assay: The ability of the exemplified anti-human TNFα antibodies MAPPs-derived peptide clusters to activate CD4+ T cells by inducing cellular proliferation was assessed. CD8+ T cells were depleted from cryopreserved PBMC's from 10 healthy donors and labeled with 1 μM Carboxyfluorescein Diacetate Succinimidyl Ester (CF SE). CD8+ T cell depleted PBMCs were seeded at 4×10⁶ cells/mL/well in AIM-V media (Life Technologies, cat #12055-083) with 5% CTS™ Immune Cell SR (Gibco, cat #A2596101) and tested in triplicate in 2.0 mL containing the different test molecules: DMSO control, media control, keyhole limpet haemocyanin (KLH; positive control), PADRE-X peptide (synthetic vaccine helper peptide, positive peptide control), or the respective anti-human TNFα antibody MAPPs-derived peptide clusters (10 μM each peptide). Cells were cultured and incubated for 7 days at 37° C. with 5% CO₂. On day 7, samples were stained with the following cell surface markers: anti-CD3, anti-CD4, anti-CD14, anti-CD19, and DAPI for viability detection by flow cytometry using a BD LSRFortessa™, equipped with a High Throughput Sampler (HTS). Data was analyzed using FlowJo® Software (FlowJo, LLC, TreeStar) and a Cellular Division Index (CDI) was calculated. Briefly, the CDI for each MAPPs-derived peptide cluster was calculated by dividing the percent of proliferating CFSE^{dim}CD4+ T cells from peptide-stimulated wells by the percent of proliferating CFSE^{dim}CD4+ T cells in the unstimulated wells. A CDI of >2.5 was considered to represent a positive response. A percent donor frequency across all donors was evaluated.

The results as demonstrated in Tables 8a and 8b, show that the LCDR1 (Table 8a) and HCDR3 (Table 8b) peptides for Ab2 induced a T cell response frequency in about 22.0% and 25% donors respectively, indicating a significantly reduced immunogenicity risk for Ab2 when compared to the positive controls. The KLH positive control induced a T cell response in 100% of donors, and the PADRE-X (Synthetic vaccine helper peptide) positive control, induced a T cell response in 67% and 62.5% of donors respectively in the two studies. This range fell within the expected range for this assay (48.1%+24.4 Positive Donor Frequency).

TABLE 8a
Frequency of CD4+ T cell responses induced by
MAPPs-derived peptides in healthy donors.

| | | Median | Median | | | Number |
|---|---|---|---|---|---|---|
| | % | CDI | CDI | | | of donors |
| Molecule | Positive | (Positive | (All | Range | | positive |
| Tested | Donors | Donors) | donors) | High | Low | (CDI > 2.5) |
| KLH | 100.0 | 190.8 | 190.8 | 1170.1 | 8.8 | 9/9 |
| PADRE-X | 67.0 | 4.0 | 3.1 | 17.8 | 0.5 | 6/9 |
| Ab2 LCDR1 | 22.0 | 5.5 | 1.1 | 6.0 | 0.6 | 2/9 |
| peptide | | | | | | |

TABLE 8b
Frequency of CD4+ T cell responses induced by
MAPPs-derived peptides in healthy donors.

| | | Median | Median | | | |
|---|---|---|---|---|---|---|
| | % | CDI | CDI | | | Number |
| Molecule | Positive | (Positive | (All | Range | | of |
| Tested | Donors | Donors) | donors) | High | Low | donors |
| KLH | 100.0 | 230.2 | 230.2 | 3558.5 | 12.0 | 8/8 |
| PADRE-X | 62.5 | 15.3 | 7.5 | 45.5 | 0.3 | 5/8 |
| Ab2 HCDR3 | 25.0 | 7.2 | 1.3 | 7.7 | 0.1 | 2/8 |
| peptide | | | | | | |

### Example 6. Biophysical Properties of Exemplified Anti-Human TNFα Antibodies

Biophysical properties of the exemplified anti-human TNFα antibodies were evaluated for developability.

Viscosity: Exemplified anti-human TNFα antibody samples were concentrated to about 125 mg/mL in a common formulation buffer matrix at pH 6. The viscosity for each antibody was measured using a VROC® initium (RheoSense) at 15° C. using the average of 9 replicate measurements. As demonstrated in Table 9, the results showed that the exemplified anti-human TNFα antibodies Ab1 (9.7 cP), Ab2 (9.2 cP), Ab3 (11.4 cP), and Ab4 (10.6 cP) had good viscosity profiles for developability.
Thermal stability: Differential Scanning calorimetry (DSC) was used to evaluate the stability of the exemplified antibodies against thermal denaturation. The thermal melting temperatures of the antibodies in PBS, pH 7.2 buffer, obtained by data fitting when unresolved, are listed in Table 9 (Tonset, TM1, TM2, and TM3). Although the thermal transitions for each domain were not all well resolved the data demonstrated in Table 9, show exemplified anti-human TNFα antibodies Ab1, Ab2, Ab3, and Ab4 had good thermal stability profiles for developability.
Aggregation upon temperature stress: The solution stability of the exemplified antibodies over time was assessed at approximately 100 mg/mL. Samples were incubated for a period of 28 days at 5° C. and 35° C. Following incubation, samples were analyzed for the percentage of high molecular weight (% HMW) species with size exclusion chromatography (SEC-HPLC). The results as demonstrated in Table 9, show exemplified anti-human TNFα antibodies Ab1, Ab2, Ab3, and Ab4 had good aggregation profiles for developability.

TABLE 9
Biophysical properties of exemplified anti-human TNFα antibodies

| | | | | | | % HMW | % HMW |
|---|---|---|---|---|---|---|---|
| Anti- | | | | | Viscosity | 28 d at | 28 d at |
| body | Tonset | TM1 | TM2 | TM3 | (cP) | 5° C. | 35° C. |
| Ab1 | 63.9 | 73.4 | 76.4 | 85.6 | 9.7 | 1.0 | 3.6 |
| Ab2 | 64.8 | 72.5 | 81.0 | 87.4 | 9.2 | 0.6 | 4.3 |
| Ab3 | 57.7 | 71.8 | 79.9 | 86.9 | 11.4 | 0.8 | 5.8 |
| Ab4 | 64.9 | 73.1 | 79.7 | 87.2 | 10.6 | 0.7 | 3.3 |

### Example 7: In Vivo Characterization of Exemplified Anti-Human TNFα Antibodies

Inhibition of human TNFα-induced CXCL1 cytokine production in vivo: Neutralization of TNFα-induced CXCL1 by the exemplified anti-human TNFα antibodies was assessed in vivo. Administration of human TNFα to C57/B6 mice induces a rapid and transient increase of mouse plasma CXCL1 levels. This allows for the interrogation of the neutralization capacity of the exemplified anti-human TNFα antibodies in vivo.

Briefly, C57/B6 mice (N=8/group) were subcutaneously administered with 0.3 mg/kg or 3 mg/kg of the exemplified antibodies or 3 mg/kg of a non-binding isotype control. Twenty-four hours post antibody administration, the mice were challenged by intraperitoneal injection of human TNFα at a dose of 3 μg/mouse. Two hours post human TNFα challenge the mice were sacrificed, blood was collected, and clarified to plasma by centrifugation. Plasma was analyzed for mouse CXCL1 concentration using a commercial MSD assay (MesoScale Discovery, P/N. K152QTG-1) according to manufacturer's instructions.

The results as demonstrated in Table 10, show that the exemplified anti-human TNFα antibodies significantly inhibited in vivo human TNFα-induced plasma CXCL1 production in a dose dependent manner, relative to isotype control treated mice (p<0.05, ANOVA followed by Turkey's Multiple Comparison test). Specifically, the exemplified anti-human TNFα antibodies inhibited TNFα induced in vivo plasma CXCL1 production by about 82% to about 93% at 3 mg/kg, and by about 46.5% to about 64.5% at 0.3 mg/kg. Thus, indicating that the exemplified anti-human TNFα antibodies neutralized the biological effects induced by human TNFα in vivo.

TABLE 10
Inhibition of human TNFα-induced CXCL1
cytokine production in vivo

| | | Plasma CXCL1 concentration | | | |
|---|---|---|---|---|---|
| | Dose | Mean | ±SEM | % | P Value |
| Antibody | mg/kg | (pg/mL) | (pg/mL) | Inhibition | vs Isotype |
| IgG1 isotype | 3 | 1013.0 | 213.1 | 0.0% | |
| control | | | | | |
| Ab1 | 3 | 185.7 | 40.1 | 83.9% | <0.0001 |
| | 0.3 | 391.6 | 81.2 | 63.0% | 0.0002 |
| Ab2 | 3 | 203.4 | 33.3 | 82.1% | <0.0001 |
| | 0.3 | 554.4 | 59.3 | 46.5% | 0.0164 |
| Ab3 | 3 | 158.9 | 32.9 | 86.6% | <0.0001 |
| | 0.3 | 378.3 | 52.8 | 64.4% | 0.0001 |
| Ab4 | 3 | 100.1 | 10.3 | 92.6% | <0.0001 |
| | 0.3 | 449.3 | 52.8 | 57.2% | 0.0009 |
| Ab5 | 3 | 182.1 | 27.8 | 84.3% | <0.0001 |
| | 0.3 | 521.7 | 136.9 | 49.8% | 0.0071 |
| IgG1 isotype | 3 | 27.3 | 9.2 | 100.0% | |
| control without | | | | | |
| TNFα IP | | | | | |
| injection | | | | | |

SEQUENCE LISTING
Ab1
SEQ ID NO: 1 HCDR1 for Ab1, Ab2, and Ab6
GYTFTGYYIH
SEQ ID NO: 2 HCDR2 for Ab1, Ab2, and Ab6
WINPYTGGTNYAQKFQG
SEQ ID NO: 3 HCDR3 for Ab1
DLYGSSNYGGDV
SEQ ID NO: 4 LCDR1 for Ab1 and Ab3
QASQGISNYLN
SEQ ID NO: 5 LCDR2 for Ab1, Ab2, Ab3, Ab4, Ab5, and Ab6
DASNLET
SEQ ID NO: 6 LCDR3 for Ab1, Ab3, and Ab5
QQYDKLPLT
SEQ ID NO: 7 VH for Ab1
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDLYGSSNY
GGDVWGQGTTVTVSS
SEQ ID NO: 8 VL for Ab1 and Ab3
DIQMTQSPSSLSASVGDRVTITCQASQGISNYLNWYQQKPGKAPKLLIYDASNLE
TGVPSRFSGSGSGTDFTFTISSLQPEDIATYYCQQYDKLPLTFGGGTKVEIK
SEQ ID NO: 9 HC for Ab1
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDLYGSSNY
GGDVWGQGTTVTVSSASTKGPCVFPLAPSSKSTSGGTAALGCLVKDYFPEPVTVS
WNSGALTSGVHTFPAVLQSSGLYSLSSVVTVPSSSLGTQTYICNVNHKPSNTKVD
KRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSD
ICVEWESNGQPENNYKTTPPVLDSDGSFFLYSKLTVDKSRWQQGNVFSCSVMHE
ALHNHYTQKSLSLSPGK
SEQ ID NO: 10 LC for Ab1 and Ab3
DIQMTQSPSSLSASVGDRVTITCQASQGISNYLNWYQQKPGKAPKLLIYDASNLE
TGVPSRFSGSGSGTDFTFTISSLQPEDIATYYCQQYDKLPLTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 11 HC DNA for Ab1
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATACAC
TGGGTGCGACAGGCCCCTGGACAAGGGCTTGAGTGGATGGGATGGATCAACC
CTTACACCGGTGGCACAAACTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATCTCTATGGTTCGAGTAA
TTACGGTGGCGACGTCTGGGGCCAAGGGACCACGGTCACCGTCTCCTCAGCT
AGCACCAAGGGCCCATGTGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCTGCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 12 LC DNA for Ab1 and Ab3
GACATCCAGATGACCCAGTCTCCATCCTCCCTGTCTGCATCTGTAGGAGACAG
AGTCACCATCACTTGCCAGGCGAGTCAGGGCATTAGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATAAGCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab2
SEQ ID NO: 1 HCDR1 for Ab1, Ab2, and Ab6
GYTFTGYYIH
SEQ ID NO: 2 HCDR2 for Ab1, Ab2, and Ab6
WINPYTGGTNYAQKFQG
SEQ ID NO: 13 HCDR3 for Ab2, Ab3, Ab4, and Ab5
DLYGSSNYGMDV
SEQ ID NO: 14 LCDR1 for Ab2, Ab4, and Ab5
QASQGIRNYLN
SEQ ID NO: 5 LCDR2 for Ab1, Ab2, Ab3, Ab4, Ab5, and Ab6
DASNLET
SEQ ID NO: 15 LCDR3 for Ab2 and Ab4
QQYDNLPLT
SEQ ID NO: 16 VH for Ab2
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDLYGSSNY
GMDVWGQGTTVTVSS
SEQ ID NO: 17 VL for Ab2 and Ab4
DIQMTQSPSSLSASVGDRVTITCQASQGIRNYLNWYQQKPGKAPKLLIYDASNLE
TGVPSRFSGSGSGTDFTFTISSLQPEDIATYYCQQYDNLPLTFGGGTKVEIK
SEQ ID NO: 18 HC for Ab2
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDLYGSSNY
GMDVWGQGTTVTVSSASTKGPCVFPLAPSSKSTSGGTAALGCLVKDYFPEPVTV
SWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPS
DICVEWESNGQPENNYKTTPPVLDSDGSFFLYSKLTVDKSRWQQGNVFSCSVMH
EALHNHYTQKSLSLSPGK
SEQ ID NO: 19 LC for Ab2 and Ab4
DIQMTQSPSSLSASVGDRVTITCQASQGIRNYLNWYQQKPGKAPKLLIYDASNLE
TGVPSRFSGSGSGTDFTFTISSLQPEDIATYYCQQYDNLPLTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 20 HC DNA for Ab2
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATACAC
TGGGTGCGACAGGCCCCTGGACAAGGGCTTGAGTGGATGGGATGGATCAACC
CTTACACCGGTGGCACAAACTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATCTCTATGGTTCGAGTAA
TTACGGTATGGACGTCTGGGGCCAAGGGACCACGGTCACCGTCTCCTCAGCT
AGCACCAAGGGCCCATGCGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCTGCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 21 LC DNA for Ab2 and Ab4
GACATCCAGATGACCCAGTCTCCATCCTCCCTGTCTGCATCTGTAGGAGACAG
AGTCACCATCACTTGCCAGGCGAGTCAGGGCATTCGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATAACCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab3
SEQ ID NO: 22 HCDR1 for Ab3, Ab4, and Ab5
GYTFTGYYMH
SEQ ID NO: 23 HCDR2 for Ab3, Ab4, and Ab5
WINPYTGGTKYAQKFQG
SEQ ID NO: 13 HCDR3 for Ab2, Ab3, Ab4, and Ab5
DLYGSSNYGMDV
SEQ ID NO: 4 LCDR1 for Ab2, Ab4, and Ab5
QASQGISNYLN
SEQ ID NO: 5 LCDR2 for Ab1, Ab2, Ab3, Ab4, Ab5, and Ab6
DASNLET
SEQ ID NO: 6 LCDR3 for Ab1, Ab3, and Ab5
QQYDKLPLT
SEQ ID NO: 24 VH for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSS
SEQ ID NO: 8 VL for Ab1 and Ab3
DIQMTQSPSSLSASVGDRVTITCQASQGISNYLNWYQQKPGKAPKLLIYDASNLE
TGVPSRFSGSGSGTDFTFTISSLQPEDIATYYCQQYDKLPLTFGGGTKVEIK
SEQ ID NO: 25 HC for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSSASTKGPCVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFY
PSDICVEWESNGQPENNYKTTPPVLDSDGSFFLYSKLTVDKSRWQQGNVFSCSV
MHEALHNHYTQKSLSLSPGK
SEQ ID NO: 10 LC for Ab1 and Ab3
DIQMTQSPSSLSASVGDRVTITCQASQGISNYLNWYQQKPGKAPKLLIYDASNLE
TGVPSRFSGSGSGTDFTFTISSLQPEDIATYYCQQYDKLPLTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 26 HC DNA for Ab3, Ab4, and Ab5
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATGCAC
TGGGTGCGACAGGCCCCTGGACAAGGGCTTGAGTGGATGGGATGGATCAACC
CTTACACCGGTGGCACAAAGTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATCTCTATGGTTCGAGTAA
TTACGGTATGGACGTCTGGGGCCAAGGGACCACGGTCACCGTCTCCTCAGCT
AGCACCAAGGGCCCATGCGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCTGCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 12 LC DNA for Ab1 and Ab3
GACATCCAGATGACCCAGTCTCCATCCTCCCTGTCTGCATCTGTAGGAGACAG
AGTCACCATCACTTGCCAGGCGAGTCAGGGCATTAGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATAAGCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab4
SEQ ID NO: 22 HCDR1 for Ab3, Ab4, and Ab5
GYTFTGYYMH
SEQ ID NO: 23 HCDR2 for Ab3, Ab4, and Ab5
WINPYTGGTKYAQKFQG
SEQ ID NO: 13 HCDR3 for Ab2, Ab3, Ab4, and Ab5
DLYGSSNYGMDV
SEQ ID NO: 14 LCDR1 for Ab2, Ab4, and Ab5
QASQGIRNYLN
SEQ ID NO: 5 LCDR2 for Ab1, Ab2, Ab3, Ab4, Ab5, and Ab6
DASNLET
SEQ ID NO: 15 LCDR3 for Ab2 and Ab4
QQYDNLPLT
SEQ ID NO: 24 VH for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSS
SEQ ID NO: 17 VL for Ab2 and Ab4
DIQMTQSPSSLSASVGDRVTITCQASQGIRNYLNWYQQKPGKAPKLLIYDASNLE
TGVPSRFSGSGSGTDFTFTISSLQPEDIATYYCQQYDNLPLTFGGGTKVEIK
SEQ ID NO: 25 HC for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSSASTKGPCVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFY
PSDICVEWESNGQPENNYKTTPPVLDSDGSFFLYSKLTVDKSRWQQGNVFSCSV
MHEALHNHYTQKSLSLSPGK
SEQ ID NO: 19 LC for Ab2 and Ab4
DIQMTQSPSSLSASVGDRVTITCQASQGIRNYLNWYQQKPGKAPKLLIYDASNLE
TGVPSRFSGSGSGTDFTFTISSLQPEDIATYYCQQYDNLPLTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 26 HC DNA for Ab3, Ab4, and Ab5
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATGCAC
TGGGTGCGACAGGCCCCTGGACAAGGGCTTGAGTGGATGGGATGGATCAACC
CTTACACCGGTGGCACAAAGTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATCTCTATGGTTCGAGTAA
TTACGGTATGGACGTCTGGGGCCAAGGGACCACGGTCACCGTCTCCTCAGCT
AGCACCAAGGGCCCATGCGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCTGCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 21 LC DNA for Ab2 and Ab4
GACATCCAGATGACCCAGTCTCCATCCTCCCTGTCTGCATCTGTAGGAGACAG
AGTCACCATCACTTGCCAGGCGAGTCAGGGCATTCGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATAACCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab5
SEQ ID NO: 22 HCDR1 for Ab3, Ab4, and Ab5
GYTFTGYYMH
SEQ ID NO: 23 HCDR2 for Ab3, Ab4, and Ab5
WINPYTGGTKYAQKFQG
SEQ ID NO: 13 HCDR3 for Ab2, Ab3, Ab4, and Ab5
DLYGSSNYGMDV
SEQ ID NO: 14 LCDR1 for Ab2, Ab4, and Ab5
QASQGIRNYLN
SEQ ID NO: 5 LCDR2 for Ab1, Ab2, Ab3, Ab4, Ab5, and Ab6
DASNLET
SEQ ID NO: 6 LCDR3 for Ab1, Ab3, and Ab5
QQYDKLPLT
SEQ ID NO: 24 VH for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSS
SEQ ID NO: 27 VL for Ab5
DIQMTQSPSSLSASVGDRVTITCQASQGIRNYLNWYQQKPGKAPKLLIYDASNLE
TGVPSRFSGSGSGTDFTFTISSLQPEDIATYYCQQYDKLPLTFGGGTKVEIK
SEQ ID NO: 25 HC for Ab3, Ab4, and Ab5
QVQLVQSGAEVKKPGASVKVSCKASGYTFTGYYMHWVRQAPGQGLEWMGWI
NPYTGGTKYAQKFQGRVTMTRDTSISTAYMELSRLRSDDTAVYYCARDLYGSSN
YGMDVWGQGTTVTVSSASTKGPCVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFY
PSDICVEWESNGQPENNYKTTPPVLDSDGSFFLYSKLTVDKSRWQQGNVFSCSV
MHEALHNHYTQKSLSLSPGK
SEQ ID NO: 28 LC for Ab5
DIQMTQSPSSLSASVGDRVTITCQASQGIRNYLNWYQQKPGKAPKLLIYDASNLE
TGVPSRFSGSGSGTDFTFTISSLQPEDIATYYCQQYDKLPLTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 26 HC DNA for Ab3, Ab4, and Ab5
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATGCAC
TGGGTGCGACAGGCCCCTGGACAAGGGCTTGAGTGGATGGGATGGATCAACC
CTTACACCGGTGGCACAAAGTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATCTCTATGGTTCGAGTAA
TTACGGTATGGACGTCTGGGGCCAAGGGACCACGGTCACCGTCTCCTCAGCT
AGCACCAAGGGCCCATGCGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCTGCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 29 LC DNA for Ab5
GACATCCAGATGACCCAGTCTCCATCCTCCCTGTCTGCATCTGTAGGAGACAG
AGTCACCATCACTTGCCAGGCGAGTCAGGGCATTCGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATAAGCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
Ab6
SEQ ID NO: 1 HCDR1 for Ab1, Ab2, and Ab6
GYTFTGYYIH
SEQ ID NO: 2 HCDR2 for Ab1, Ab2, and Ab6
WINPYTGGTNYAQKFQG
SEQ ID NO: 30 HCDR3 for Ab6
DIYGSSNYGGDV
SEQ ID NO: 31 LCDR1 for Ab6
QASQDISNYLN
SEQ ID NO: 5 LCDR2 for Ab1, Ab2, Ab3, Ab4, Ab5, and Ab6
DASNLET
SEQ ID NO: 32 LCDR3 for Ab6
QQYDTLPLT
SEQ ID NO: 33 VH for Ab6
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDIYGSSNY
GGDVWGQGTTVTVSS
SEQ ID NO: 34 VL for Ab6
DIQMTQSPSSLSASVGDRVTITCQASQDISNYLNWYQQKPGKAPKLLIYDASNLE
TGVPSRFSGSGSGTDFTFTISSLQPEDIATYYCQQYDTLPLTFGGGTKVEIK
SEQ ID NO: 35 HC for Ab6
QVQLVQSGAEVKKPGASVKVSCKASGYTFTGYYIHWVRQAPGQGLEWMGWIN
PYTGGTNYAQKFQGRVTMTRDTSISTAYMELSRLRSDDTAVYYCARDIYGSSNY
GGDVWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KRVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHE
ALHNHYTQKSLSLSPGK
SEQ ID NO: 36 LC for Ab6
DIQMTQSPSSLSASVGDRVTITCQASQDISNYLNWYQQKPGKAPKLLIYDASNLE
TGVPSRFSGSGSGTDFTFTISSLQPEDIATYYCQQYDTLPLTFGGGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 37 HC DNA for Ab6
CAGGTGCAGCTGGTGCAGTCTGGGGCTGAGGTGAAGAAGCCTGGGGCCTCAG
TGAAGGTCTCCTGCAAGGCTTCTGGATACACCTTCACCGGCTACTATATACAC
TGGGTGCGACAGGCCCCTGGACAAGGGCTTGAGTGGATGGGATGGATCAACC
CTTACACCGGTGGCACAAACTATGCACAGAAGTTTCAGGGCAGGGTCACCAT
GACCAGGGACACGTCCATCAGCACAGCCTACATGGAGCTGAGCAGGCTGAGA
TCTGACGACACGGCCGTGTATTACTGTGCGAGAGATATCTATGGTTCGAGTAA
TTACGGTGGCGACGTCTGGGGCCAAGGGACCACGGTCACCGTCTCCTCAGCT
AGCACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCT
TCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCA
ACTGGTATGTGGACGGCGTGGAGGTGCATAATGCCAAGACAAAGCCGCGGGA
GGAGCAGTACAACAGCACGTACCGTGTGGTCAGCGTCCTCACCGTCCTGCAC
CAAGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGA
ACCACAGGTGTACACCCTGCCCCCATCCCGGGAGGAGATGACCAAGAACCAA
GTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGT
GCTGGACTCCGACGGCTCCTTCTTCCTCTATTCCAAGCTCACCGTGGACAAGA
GCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 38 LC DNA for Ab6
GACATCCAGATGACCCAGTCTCCATCCTCCCTGTCTGCATCTGTAGGAGACAG
AGTCACCATCACTTGCCAGGCGAGTCAGGACATTAGCAACTATTTAAATTGGT
ATCAGCAGAAACCAGGGAAAGCCCCTAAGCTCCTGATCTACGATGCATCCAA
TTTGGAAACAGGGGTCCCATCAAGGTTCAGTGGAAGTGGATCTGGGACAGAT
TTTACTTTCACCATCAGCAGCCTGCAGCCTGAAGATATTGCAACATATTACTG
TCAACAGTATGATACCCTCCCGCTCACTTTCGGCGGAGGGACCAAGGTGGAG
ATCAAACGGACCGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGC
SEQ ID NO: 39 Human TNFα protein
MSTESMIRDVELAEEALPKKTGGPQGSRRCLFLSLFSFLIVAGATTLFCLLHFGVI
GPQREEFPRDLSLISPLAQAVRSSSRTPSDKPVAHVVANPQAEGQLQWLNRRANA
LLANGVELRDNQLVVPSEGLYLIYSQVLFKGQGCPSTHVLLTHTISRIAVSYQTKV
NLLSAIKSPCQRETPEGAEAKPWYEPIYLGGVFQLEKGDRLSAEINRPDYLDFAES
GQVYFGIIAL
SEQ ID NO: 40 Rhesus macaque TNFα protein
MSTESMIRDVELAEEALPRKTAGPQGSRRCWFLSLFSFLLVAGATTLFCLLHFGVI
GPQREEFPKDPSLISPLAQAVRSSSRTPSDKPVAHVVANPQAEGQLQWLNRRANA
LLANGVELTDNQLVVPSEGLYLIYSQVLFKGQGCPSNHVLLTHTISRIAVSYQTK
VNLLSAIKSPCQRETPEGAEAKPWYEPIYLGGVFQLEKGDRLSAEINLPDYLDFAE
SGQVYFGIIAL
SEQ ID NO: 41 Mouse TNFα protein
MSTESMIRDVELAEEALPQKMGGFQNSRRCLCLSLFSFLLVAGATTLFCLLNFGVI
GPQRDEKFPNGLPLISSMAQTLTLRSSSQNSSDKPVAHVVANHQVEEQLEWLSQR
ANALLANGMDLKDNQLVVPADGLYLVYSQVLFKGQGCPDYVLLTHTVSRFAIS
YQEKVNLLSAVKSPCPKDTPEGAELKPWYEPIYLGGVFQLEKGDQLSAEVNLPK
YLDFAESGQVYFGVIAL
SEQ ID NO: 42 Rat TNFα protein
MSTESMIRDVELAEEALPKKMGGLQNSRRCLCLSLFSFLLVAGATTLFCLLNFGV
IGPNKEEKFPNGLPLISSMAQTLTLRSSSQNSSDKPVAHVVANHQAEEQLEWLSQ
RANALLANGMDLKDNQLVVPADGLYLIYSQVLFKGQGCPDYVLLTHTVSRFAIS
YQEKVSLLSAIKSPCPKDTPEGAELKPWYEPMYLGGVFQLEKGDLLSAEVNLPK
YLDITESGQVYFGVIAL
SEQ ID NO: 43 Rabbit TNFα protein
MSTESMIRDVELAEGPLPKKAGGPQGSKRCLCLSLFSFLLVAGATTLFCLLHFRVI
GPQEEESPNNLHLVNPVAQMVTLRSASRALSDKPLAHVVANPQVEGQLQWLSQ
RANALLANGMKLTDNQLVVPADGLYLIYSQVLFSGQGCRSYVLLTHTVSRFAVS
YPNKVNLLSAIKSPCHRETPEEAEPMAWYEPIYLGGVFQLEKGDRLSTEVNQPEY
LDLAE SGQVYFGIIAL
SEQ ID NO: 44 Canine TNFα protein
MSTESMIRDVELAEEPLPKKAGGPPGSRRCFCLSLFSFLLVAGATTLFCLLHFGVI
GPQREELPNGLQLISPLAQTVKSSSRTPSDKPVAHVVANPEAEGQLQWLSRRANA
LLANGVELTDNQLIVPSDGLYLIYSQVLFKGQGCPSTHVLLTHTISRFAVSYQTKV
NLLSAIKSPCQRETPEGTEAKPWYEPIYLGGVFQLEKGDRLSAEINLPNYLDFAES
GQVYFGIIAL
SEQ ID NO: 45 Cynomolgus monkey TNFα protein
MSTESMIQDVELAEEALPRKTAGPQGSRRCWFLSLFSFLLVAGAATLFCLLHFGV
IGPQREEFPKDPSLISPLAQAVRSSSRTPSDKPVAHVVANPQAEGQLQWLNRRAN
ALVANGVELTDNQLVVPSEGLYLIYSQVLFKGQGCPSNHVLLTHTISRIAVSYQT
KVNLLSAIKSPCQRETPEGAEAKPWYEPIYLGGVFQLEKGDRLSAEINLPDYLDFA
ESGQVYFGIIAL
SEQ ID NO: 46 LCDR1 consensus sequence
QASQGIXaa₇NYLN
Wherein Xaa₇ is Serine or Arginine
SEQ ID NO: 47 LCDR3 consensus sequence
QQYDXaa₅LPLT
Wherein Xaa5 is Asparagine or Lysine
SEQ ID NO: 48 human TNFR1
MGLSTVPDLLLPLVLLELLVGIYPSGVIGLVPHLGDREKRDSVCPQGKYIHPQNNS
ICCTKCHKGTYLYNDCPGPGQDTDCRECESGSFTASENHLRHCLSCSKCRKEMG
QVEISSCTVDRDTVCGCRKNQYRHYWSENLFQCFNCSLCLNGTVHLSCQEKQNT
VCTCHAGFFLRENECVSCSNCKKSLECTKLCLPQIENVKGTEDSGTTVLLPLVIFF
GLCLLSLLFIGLMYRYQRWKSKLYSIVCGKSTPEKEGELEGTTTKPLAPNPSFSPT
PGFTPTLGFSPVPSSTFTSSSTYTPGDCPNFAAPRREVAPPYQGADPILATALASDPI
PNPLQKWEDSAHKPQSLDTDDPATLYAVVENVPPLRWKEFVRRLGLSDHEIDRL
ELQNGRCLREAQYSMLATWRRRTPRREATLELLGRVLRDMDLLGCLEDIEEALC
GPAALPPAPSLLR
SEQ ID NO: 49 human TNFR2
MAPVAVWAALAVGLELWAAAHALPAQVAFTPYAPEPGSTCRLREYYDQTAQM
CCSKCSPGQHAKVFCTKTSDTVCDSCEDSTYTQLWNWVPECLSCGSRCSSDQVE
TQACTREQNRICTCRPGWYCALSKQEGCRLCAPLRKCRPGFGVARPGTETSDVV
CKPCAPGTFSNTTSSTDICRPHQICNVVAIPGNASMDAVCTSTSPTRSMAPGAVHL
PQPVSTRSQHTQPTPEPSTAPSTSFLLPMGPSPPAEGSTGDFALPVGLIVGVTALGL
LIIGVVNCVIMTQVKKKPLCLQREAKVPHLPADKARGTQGPEQQHLLITAPSSSSS
SLESSASALDRRAPTRNQPQAPGVEASGAGEARASTGSSDSSPGGHGTQVNVTCI
VNVCSSSDHSSQCSSQASSTMGDTDSSPSESPKDEQVPFSKEECAFRSQLETPETL
LGSTEEKPLPLGVPDAGMKPS

1. An antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1;
the HCDR2 comprises SEQ ID NO: 2;
the HCDR3 comprises SEQ ID NO: 3;
the LCDR1 comprises SEQ ID NO: 4;
the LCDR2 comprises SEQ ID NO: 5; and
the LCDR3 comprises SEQ ID NO: 6.

2. The antibody claim 1, wherein the VH comprises SEQ ID NO: 7 and the VL comprises SEQ ID NO: 8.

3. The antibody of claim 1, wherein the antibody comprises a heavy chain (HC) and a light chain (LC), wherein the HC comprises SEQ ID NO: 9 and the LC comprises SEQ ID NO: 10.

4. An antibody that binds human TNFα, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
a. the HCDR1 comprises SEQ ID NO: 22;
the HCDR2 comprises SEQ ID NO: 23;
the HCDR3 comprises SEQ ID NO: 13;
the LCDR1 comprises SEQ ID NO: 4, 14, or 46;
the LCDR2 comprises SEQ ID NO: 5; and
the LCDR3 comprises SEQ ID NO: 6;
b. the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15 or 47;
c. the HCDR1 comprises SEQ ID NO: 1;
the HCDR2 comprises SEQ ID NO: 2;
the HCDR3 comprises SEQ ID NO: 30;
the LCDR1 comprises SEQ ID NO: 31;
the LCDR2 comprises SEQ ID NO: 5; and
the LCDR3 comprises SEQ ID NO: 32; or
d. the HCDR1 comprises SEQ ID NO: 1;
the HCDR2 comprises SEQ ID NO: 2;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15.

5. The antibody of claim 4, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 4;
the LCDR2 comprises SEQ ID NO: 5; and
the LCDR3 comprises SEQ ID NO: 6.

6. The antibody of claim 4, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 6.

7. The antibody of claim 4, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15.

8. The antibody of claim 4, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 46;
the LCDR2 comprises SEQ ID NO: 5; and
the LCDR3 comprises SEQ ID NO: 6.

9. The antibody of claim 4, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 47.

10. The antibody of claim 4, wherein the VH comprises SEQ ID NO: 24 and the VL comprises SEQ ID NO: 8, 17, or 27.

11. The antibody of claim 4, wherein the antibody comprises a heavy chain (HC) and a light chain (LC), wherein the HC comprises SEQ ID NO: 25 and the LC comprises SEQ ID NO: 10, 19, or 28.

12. The antibody of claim 4, wherein:
the HCDR1 comprises SEQ ID NO: 1;
the HCDR2 comprises SEQ ID NO: 2;
the HCDR3 comprises SEQ ID NO: 30;
the LCDR1 comprises SEQ ID NO: 31;
the LCDR2 comprises SEQ ID NO: 5; and
the LCDR3 comprises SEQ ID NO: 32.

13. The antibody of claim 12, wherein the VH comprises SEQ ID NO: 33 and the VL comprises SEQ ID NO: 34.

14. The antibody of claim 12, wherein the antibody comprises a heavy chain (HC) and a light chain (LC), wherein the HC comprises SEQ ID NO: 35 and the LC comprises SEQ ID NO: 36.

15. The antibody of claim 4, wherein:
the HCDR1 comprises SEQ ID NO: 1;
the HCDR2 comprises SEQ ID NO: 2;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15.

16. The antibody of claim 15, wherein the VH comprises SEQ ID NO: 16 and the VL comprises SEQ ID NO: 17.

17. The antibody of claim 15, wherein the antibody comprises a heavy chain (HC) and a light chain (LC), wherein the HC comprises SEQ ID NO: 18 and the LC comprises SEQ ID NO: 19.

18. The antibody of claim 1, wherein the antibody comprises a light chain and a heavy chain, wherein the heavy chain comprises:
a cysteine at amino acid residue 124 (EU numbering);
a cysteine at amino acid residue 378 (EU numbering); or
a cysteine at amino acid residue 124 (EU numbering) and a cysteine at amino acid residue 378 (EU numbering).

19. The antibody of claim 4, wherein the antibody comprises a light chain and a heavy chain, wherein the heavy chain comprises:
a cysteine at amino acid residue 124 (EU numbering);
a cysteine at amino acid residue 378 (EU numbering); or
a cysteine at amino acid residue 124 (EU numbering) and a cysteine at amino acid residue 378 (EU numbering).

20. The antibody of claim 1, wherein the antibody comprises a heavy chain (HC) and a light chain (LC), wherein the HC is human IgG1 isotype.

21. The antibody of claim 4, wherein the antibody comprises a heavy chain (HC) and a light chain (LC), wherein the HC is human IgG1 isotype.

22. A nucleic acid comprising a sequence encoding SEQ ID NO: 9, 10, 18, 19, 25, 28, 35 or 36.

23. A vector comprising the nucleic acid of claim 22.

24. The vector of claim 23, wherein the vector comprises a first nucleic acid sequence encoding SEQ ID NO: 9, 18, 25, or 35 and a second nucleic acid sequence encoding SEQ ID NO: 10, 19, 28, or 36.

25. The vector of claim 23, wherein the vector comprises a first nucleic acid sequence encoding SEQ ID NO: 9 and a second nucleic acid sequence encoding SEQ ID NO: 10.

26. The vector of claim 23, wherein the vector comprises a first nucleic acid sequence encoding SEQ ID NO: 18 and a second nucleic acid sequence encoding SEQ ID NO: 19.

27. The vector of claim 23, wherein the vector comprises a first nucleic acid sequence encoding SEQ ID NO: 25 and a second nucleic acid sequence encoding SEQ ID NO: 10, 19, or 28.

28. The vector of claim 23, wherein the vector comprises a first nucleic acid sequence encoding SEQ ID NO: 35 and a second nucleic acid sequence encoding SEQ ID NO: 36.

29. A composition comprising a first vector comprising a nucleic acid sequence encoding SEQ ID NO: 9, 18, 25, or 35 and a second vector comprising a nucleic acid sequence encoding SEQ ID NO: 10, 19, 28, or 36.

30. The composition of claim 29, wherein the first vector comprises a nucleic acid sequence encoding SEQ ID NO: 9 and the second vector comprises a nucleic acid sequence encoding SEQ ID NO: 10.

31. The composition of claim 29, wherein the first vector comprises a nucleic acid sequence encoding SEQ ID NO: 18, and the second vector comprises a nucleic acid sequence encoding SEQ ID NO: 19.

32. The composition of claim 29, wherein the first vector comprises a nucleic acid sequence encoding SEQ ID NO: 25, and the second vector comprises a nucleic acid sequence encoding SEQ ID NO: 10, 19, or. 28.

33. The composition of claim 29, wherein the first vector comprises a nucleic acid sequence encoding SEQ ID NO: 35, and the second vector comprises a nucleic acid sequence encoding SEQ ID NO: 36.

34. A cell comprising the vector of claim 23.

35. The cell of claim 34, wherein the cell is a mammalian cell.

36. A process of producing an antibody comprising culturing the cell of claim 34, under conditions such that the antibody is expressed and recovering the expressed antibody from the culture medium.

37. An antibody produced by the process of claim 36.

38. An antibody drug conjugate comprising the antibody of claim 1.

39. An antibody drug conjugate comprising the antibody of claim 4.

40. A pharmaceutical composition comprising the antibody of claim 1, and a pharmaceutically acceptable excipient, diluent, or carrier.

41. A pharmaceutical composition comprising the antibody of claim 4, and a pharmaceutically acceptable excipient, diluent, or carrier.

42. A method of treating a TNFα associated disorder in a subject in need thereof, comprising administering to the subject a therapeutically effective amount of the antibody of claim 1.

43. The method of claim 42, wherein the TNFα associated disorder is a chronic autoinflammatory immune disorder.

44. The method of claim 43, wherein the chronic autoinflammatory immune disorder is selected from Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis, or Behcet's Disease.

45. The method claim 42, wherein the subject being administered the therapeutically effective amount of the antibody received a prior treatment with other anti-TNFα therapeutic, and wherein the subject developed anti-drug antibodies against the other anti-TNFα therapeutic.

46. The method of claim 45, wherein the other anti-TNFα therapeutic is selected from Adalimumab, Infliximab, Golimumab, Certolizumab, or Etanercept.

47. The method of claim 45, wherein the antibody has low to no binding to anti-drug antibodies against Adalimumab.

48. A method of treating a TNFα associated disorder in a subject in need thereof, comprising administering to the subject a therapeutically effective amount of the antibody of claim 4.

49. The method of claim 48, wherein the TNFα associated disorder is a chronic autoinflammatory immune disorder.

50. The method of claim 49, wherein the chronic autoinflammatory immune disorder is selected from Rheumatoid Arthritis (RA), Juvenile Idiopathic Arthritis, Psoriatic Arthritis (PsA), Ankylosing Spondylitis (AS), Crohn's Disease (CD), Ulcerative Colitis, Plaque Psoriasis (PS), Hidradenitis Suppurativa, Uveitis, Non-Infectious Intermediate, Posterior, Pan Uveitis, or Behcet's Disease.

51. The method claim 48, wherein the subject being administered the therapeutically effective amount of the antibody received a prior treatment with other anti-TNFα therapeutic, and wherein the subject developed anti-drug antibodies against the other anti-TNFα therapeutic.

52. The method of claim 51, wherein the other anti-TNFα therapeutic is selected from Adalimumab, Infliximab, Golimumab, Certolizumab, or Etanercept.

53. The method of claim 51, wherein the antibody has low to no binding to anti-drug antibodies against Adalimumab.

54. The antibody of claim 1, wherein the antibody neutralizes human TNFα.

55. The antibody of claim 4, wherein the antibody neutralizes human TNFα.

56. The antibody of claim 1, wherein the antibody is an internalizing antibody.

57. The antibody of claim 4, wherein the antibody is an internalizing antibody.

58. The antibody of claim 1, wherein the antibody has low immunogenicity.

59. The antibody of claim 4, wherein the antibody has low immunogenicity.


SEQUENCE LISTING (49 sequences)

SEQ ID NO: 1 - protein, 10 aa, synthetic construct
GYTFTGYYIH

SEQ ID NO: 2 - protein, 17 aa, synthetic construct
WINPYTGGTNYAQKFQG

SEQ ID NO: 3 - protein, 12 aa, synthetic construct
DLYGSSNYGGDV

SEQ ID NO: 4 - protein, 11 aa, synthetic construct
QASQGISNYLN

SEQ ID NO: 5 - protein, 7 aa, synthetic construct
DASNLET

SEQ ID NO: 6 - protein, 9 aa, synthetic construct
QQYDKLPLT

SEQ ID NO: 7 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGGDVWGQGTTVTVSS

SEQ ID NO: 8 - protein, 107 aa, synthetic construct
DIQMTQSPSSLSASVGDRVTITCQASQGISNYLNWYQQKPGKAPKLLIYDASNLETGVPSRFSGSGSGTDFTFTISSLQPEDIATYYCQQYDKLPLTFGGGTKVEIK

SEQ ID NO: 9 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGGDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCQASQGISNYLNWYQQKPGKAPKLLIYDASNLETGVPSRFSGSGSGTDFTFTISSLQPEDIATYYCQQYDKLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatctctatggttcgagtaattacggtggcgacgtctggggccaagggaccacggtcaccgtctcctcagctagcaccaagggcccatgtgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 12 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataagctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 13 - protein, 12 aa, synthetic construct
DLYGSSNYGMDV

SEQ ID NO: 14 - protein, 11 aa, synthetic construct
QASQGIRNYLN

SEQ ID NO: 15 - protein, 9 aa, synthetic construct
QQYDNLPLT

SEQ ID NO: 16 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGMDVWGQGTTVTVSS

SEQ ID NO: 17 - protein, 107 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDNLPLTFGGGTKVEIK

SEQ ID NO: 18 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGMDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatctctatggttcgagtaattacggtatggacgtctggggccaagggaccacggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 21 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattcgcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataacctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 22 - protein, 10 aa, synthetic construct
GYTFTGYYMH

SEQ ID NO: 23 - protein, 17 aa, synthetic construct
WINPYTGGTKYAQKFQG

SEQ ID NO: 24 - protein, 121 aa, synthetic construct
QVQLVQSGAEVKKPGASVKVSCKASGYTFTGYYMHWVRQAPGQGLEWMGWINPYTGGTKYAQKFQGRVTMTRDTSISTAYMELSRLRSDDTAVYYCARDLYGSSNYGMDVWGQGTTVTVSS

SEQ ID NO: 25 - protein, 451 aa, synthetic construct
QVQLVQSGAEVKKPGASVKVSCKASGYTFTGYYMHWVRQAPGQGLEWMGWINPYTGGTKYAQKFQGRVTMTRDTSISTAYMELSRLRSDDTAVYYCARDLYGSSNYGMDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 26 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttacaccggtggcacaaagtatgcacagaagtttcagggcagggtcaccatgaccagggacacgtccatcagcacagcctacatggagctgagcaggctgagatctgacgacacggccgtgtattactgtgcgagagatctctatggttcgagtaattacggtatggacgtctggggccaagggaccacggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 27 - protein, 107 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDKLPLTFGGGTKVEIK

SEQ ID NO: 28 - protein, 214 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDKLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattcgcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataagctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 30 - protein, 12 aa, synthetic construct
DIYGSSNYGGDV

SEQ ID NO: 31 - protein, 11 aa, synthetic construct
QASQDISNYLN

SEQ ID NO: 32 - protein, 9 aa, synthetic construct
QQYDTLPLT

SEQ ID NO: 33 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDIYGSSNYGGDVWGQGTTVTVSS

SEQ ID NO: 34 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDTLPLTFGGGTKVEIK

SEQ ID NO: 35 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDIYGSSNYGGDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDTLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatatctatggttcgagtaattacggtggcgacgtctggggccaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 38 - DNA, 642 bp, synthetic construct
  source 1..642: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataccctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 39 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 40 - protein, 233 aa, Macaca mulatta
MSTESMIRDVELAEEALPRKTAGPQGSRRCWFLSLFSFLLVAGATTLFCLLHFGVIGPQREEFPKDPSLISPLAQAVRSSSRTPSDKPVAHVVANPQAEGQLQWLNRRANALLANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 41 - protein, 235 aa, Mus musculus
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 42 - protein, 235 aa, Rattus norvegicus
MSTESMIRDVELAEEALPKKMGGLQNSRRCLCLSLFSFLLVAGATTLFCLLNFGVIGPNKEEKFPNGLPLISSMAQTLTLRSSSQNSSDKPVAHVVANHQAEEQLEWLSQRANALLANGMDLKDNQLVVPADGLYLIYSQVLFKGQGCPDYVLLTHTVSRFAISYQEKVSLLSAIKSPCPKDTPEGAELKPWYEPMYLGGVFQLEKGDLLSAEVNLPKYLDITESGQVYFGVIAL

SEQ ID NO: 43 - protein, 234 aa, Oryctolagus cuniculus
MSTESMIRDVELAEGPLPKKAGGPQGSKRCLCLSLFSFLLVAGATTLFCLLHFRVIGPQEEESPNNLHLVNPVAQMVTLRSASRALSDKPLAHVVANPQVEGQLQWLSQRANALLANGMKLTDNQLVVPADGLYLIYSQVLFSGQGCRSYVLLTHTVSRFAVSYPNKVNLLSAIKSPCHRETPEEAEPMAWYEPIYLGGVFQLEKGDRLSTEVNQPEYLDLAESGQVYFGIIAL

SEQ ID NO: 44 - protein, 233 aa, Canis lupus
MSTESMIRDVELAEEPLPKKAGGPPGSRRCFCLSLFSFLLVAGATTLFCLLHFGVIGPQREELPNGLQLISPLAQTVKSSSRTPSDKPVAHVVANPEAEGQLQWLSRRANALLANGVELTDNQLIVPSDGLYLIYSQVLFKGQGCPSTHVLLTHTISRFAVSYQTKVNLLSAIKSPCQRETPEGTEAKPWYEPIYLGGVFQLEKGDRLSAEINLPNYLDFAESGQVYFGIIAL

SEQ ID NO: 45 - protein, 233 aa, Macaca fascicularis
MSTESMIQDVELAEEALPRKTAGPQGSRRCWFLSLFSFLLVAGAATLFCLLHFGVIGPQREEFPKDPSLISPLAQAVRSSSRTPSDKPVAHVVANPQAEGQLQWLNRRANALVANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 46 - protein, 11 aa, synthetic construct
  SITE 7: note = X at position 7 is Serine or Arginine
QASQGIXNYLN

SEQ ID NO: 47 - protein, 9 aa, synthetic construct
  SITE 5: note = X at position 5 is Asparagine or Lysine
QQYDXLPLT

SEQ ID NO: 48 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 49 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS